(19) United States
(12) Patent Application Publication
(10) Pub. No.: US 2023/0084300 A1
(43) Pub. Date: Mar. 16, 2023

(54) PREVENTION AND TREATMENT OF INFECTIONS INCLUDING THOSE CAUSED BY CORONAVIRUS

(71) Applicant: IMMODULON THERAPEUTICS LIMITED, London (GB)

(72) Inventors: Glen MARTYN, Greater London (GB); Jakob KAMPINGA, Greater London (GB); Thomas KLEEN, Greater London (GB)

(21) Appl. No.: 17/904,155

(22) PCT Filed: Feb. 12, 2021
(86) PCT No.: PCT/GB2021/050354

§ 371 Date: Aug. 12, 2022

Related U.S. Application Data
(60) Provisional application No. 62/976,792, filed on Feb. 14, 2020.

(30) Foreign Application Priority Data
Mar. 19, 2020 (GB) 2004007.7
May 13, 2020 (GB) 2007045.4

(51) Int. Cl. A61K 39/04; A61K 39/39

(52) U.S. Cl. CPC A61K 39/04; A61K 39/39; A61K 2039/545

(57) ABSTRACT

The present invention relates to the use of an immunomodulator comprising non-pathogenic non-viable Mycobacterium, such as Mycobacterium obuense (IMM-101), and one or more biologically-active agents, suitably an antigen or antigenic determinant, in a method of treating or preventing a infection and/or the symptoms associated thereof in a human subject at elevated risk of exposure to and/or severity of said infection, such as a healthcare or social care worker or cancer patient, wherein said viral infections are preferably caused by a coronavirus. The invention is particularly applicable to those infections caused by SARS-CoV, MERS-CoV, or SARS-CoV-2 (COVID-19). The invention also provides an adjuvant and pharmaceutical composition comprising said immunomodulator optionally with a pharmaceutically acceptable carrier, diluent or excipient, as well as the use of said adjuvant or said pharmaceutical composition in the manufacture of a medicament for the treatment or preventions of said infections.

### FIELD OF INVENTION

The present invention relates to the treatment or prevention of infections caused by a virus, bacteria, protozoa or fungus, in a human subject at elevated risk of exposure to and/or severity of said infection, in particular viral infections caused by the SARS-Cov-2 coronavirus

### BACKGROUND

Since their initial discovery in the 1960s, a variety of human-infecting coronaviruses have been characterised. From the family Coronaviridae, these viruses primarily infect the upper respiratory and gastrointestinal tracts to cause respiratory infections. Whilst many such infections are mild and routinely include the common cold, for example, far more pathogenic and potentially lethal strains exist, including SARS, MERS, and the 2019 outbreak strain of SARS-CoV-2, (2019-nCov or COVID-19).

SARS (Severe Acute Respiratory Syndrome), MERS (Middle East Respiratory Syndrome) and SARS-CoV-2 (SARS-related Coronavirus 2) are all highly pathogenic human coronaviruses responsible for acute and chronic diseases of the respiratory, hepatic, gastrointestinal and neurological systems. It is thought that each virus emerged from animal reservoirs to result in human epidemics, with the outbreak of SARS in 2002, MERS in 2012, and SARS-CoV-2 in late 2019 the latter more commonly known as COVID-19.

Coronaviruses are enveloped single-stranded RNA viruses named so for their crown-like surface structure composed of spike (S), envelope (E), membrane (M) and nucleocapsid (N) proteins. The spike protein in particular is responsible for the action of entering a host cell, wherein the coronavirus is able to transcribe its RNA for intracytoplasmic replication. Indeed, coronaviruses have a unique ability to replicate and survive in the intracellular space of a macrophage, whereby multiple encoded interferon antagonists are thought to hinder the activation of type I interferon (IFN) and interferon stimulated genes (ISGs), dampening the host immune response and contributing to the resultant pathogenesis of the virus (Rose et al. 2010, Journal of Virology 84 (11): 5656-5669).

Upon genome replication and polyprotein formation, the viruses assemble and are released from the infected cell to further disseminate. Transmission between hosts is considered to occur primarily by contact with respiratory droplets infected with such viral particles, generated through sneezing and coughing (CDC.gov, 2020).

Coronaviruses can emerge from animal reservoirs to cause significant epidemics in humans, as exemplified by Severe Acute Respiratory Syndrome coronavirus (SARS-CoV) in 2002-2003 and Middle East Respiratory Syndrome coronavirus (MERS-CoV), which was recognized as an emerging virus in 2012, each of which resulted in over 8000 infections and 774 deaths, and 2500 infections and 862 deaths, per respective outbreak (WHO, 2020). Declared a global emergency by the World Health Organisation (WHO), the newly discovered and rapidly disseminating SARS-CoV-2, sharing ˜70% genetic similarity to the SARS-CoV, is likely to have similar epidemiological characteristics and thus presents a pressing area of healthcare concern. Crucially, there were no vaccines or antiviral drugs suitable for the prevention or treatment of human coronavirus infections at the time of first preparing the application as first filed (Habibzadeh & Stoneman 2020, Int J Occup Environ Med 11 (2): 65-71). In the intervening months, several vaccines and anti-viral agents have been approved in various countries, many under emergency use provisions.

A SARS-CoV-2/COVID-19 health emergency challenge is evidence of a lack of effective virus-specific treatments or vaccines, which thus leads to a high unmet need for the protection of high-risk populations, including health care workers and patients in acute danger of nosocomial transmission of SARS-CoV-2, or in other confined spaces, such as during quarantine settings.

At the time of first writing, according to one database there are over 1,340 interventional trials planned or ongoing by more than 440 companies and institutions across 50 countries, with 70% of agents involving repurposed drugs but only 12% of trials are for prevention. More recently, the number of pipeline drugs stands at 1,527 with 4,241 clinical trials ongoing, involving some 1,192 companies and institutions driving prophylactic and curative innovations.

Despite this, there still exists a particular need for a simple, safe and broadly effective method of treating or preventing infections or symptoms thereof caused by a coronavirus, in particular those related to SARS-CoV-2.

### Description of Figures

FIGS. 1a to d—an investigation into the influence of specific heat-killed mycobacteria on responses in human γδ T-cell-cells, including heat-killed M. vaccae NCTC 11659, M. obuense NCTC 11365 and BCG (Danish Strain 1331).

### SUMMARY OF INVENTION

This invention is based on the surprising discovery that administration to a human subject of a Mycobacterial composition, such as Mycobacterium obuense, can result in an effective treatment for infections caused by a virus, bacteria, protozoa or fungus, particularly where the infection is viral and caused by a coronavirus, including SARS-CoV, MERS-CoV, and the recent SARS-CoV-2. Furthermore, such a Mycobacterium administration can also have prophylactic effect within human subjects, hence administering an immunomodulator comprising M. obuense, for example, can also surprisingly be used as a preventative therapy against infections caused by these infectious agents. The immunomodulator can also be used to treat symptoms associated with such infections.

In a first aspect of the invention, there is provided a method of treating or preventing an infection and/or the symptoms thereof in a human subject at elevated risk of exposure to and/or severity of said infection, said method comprising administering to the human subject an immunomodulator, wherein the immunomodulator comprises a non-pathogenic non-viable Mycobacterium and wherein the infection is an infection caused by a virus, bacteria, protozoa or fungus, preferably wherein the virus is a coronavirus.

In a second aspect of the invention, there is provided an immunomodulator for use in the treatment or prevention of an infection and/or the immunological abnormalities accompanying said infection in a human subject at elevated risk of exposure to and/or severity of said infection, wherein the immunomodulator comprises a non-pathogenic non-viable Mycobacterium, and wherein the infection is an infection caused by a virus, bacteria, protozoa or fungus, preferably wherein the virus is a coronavirus.

In a third aspect of the invention, there is provided a pharmaceutical composition comprising a non-pathogenic non-viable Mycobacterium and optionally a pharmaceutically acceptable carrier, diluent or excipient, for use in modifying a cellular immune response in a human subject infected with a virus, bacteria, protozoa or fungus, preferably wherein the virus is a coronavirus, and/or said human subject is at elevated risk of exposure to and/or severity of said infection.

In a fourth aspect of the invention, there is provided a use of an immunomodulator or pharmaceutical composition, as defined in the second or third aspects, in the manufacture of a medicament for the treatment or prevention of an infection and/or the symptoms thereof in a human subject at elevated risk of exposure to and/or severity of said infection, wherein the infection is an infection caused by a virus, bacteria, protozoa or fungus, preferably wherein the virus is a coronavirus.

In a fifth aspect of the invention, there is provided a method of treating or preventing a infection and/or the symptoms thereof in a human subject at elevated risk of exposure to and/or severity of said infection, said method comprising administering to the human subject, an immunomodulator and one or more biologically-active agents, wherein the immunomodulator comprises a non-pathogenic non-viable Mycobacterium, and wherein the infection is an infection caused by a virus, bacteria, protozoa or fungus, preferably wherein the virus is a coronavirus.

In a sixth aspect of the invention, there is provided an adjuvant for use in conjunction with one or more biologically-active agents in the treatment or prevention of an infection and/or the symptoms thereof in a human subject at elevated risk of exposure to and/or severity of said infection, wherein the adjuvant comprises a non-pathogenic non-viable Mycobacterium, and wherein the infection is an infection caused by a virus, bacteria, protozoa or fungus, preferably wherein the virus is a coronavirus.

In a seventh aspect of the invention, there is provided method or adjuvant according to any preceding aspects, wherein the adjuvant is administered to a human subject at elevated risk of exposure to and/or severity of said viral infection for the treatment and/or prevention of an infection caused by a virus, bacteria, protozoa or fungus, preferably wherein the virus is a coronavirus, by; (i), eliciting a Type 1 interferon response, and/or; (ii), enhancing the expression of one or more pro-inflammatory cytokines, and/or; (iii), eliciting trained immunity in the human subject, optionally wherein the adjuvant comprises the non-pathogenic non-viable Mycobacterium obuense strain deposited under the Budapest Treaty under accession number NCTC 13365

In an eighth aspect of the invention, there is provided a pharmaceutical composition comprising a non-pathogenic non-viable Mycobacterium and one or more biologically-active agents, and optionally a pharmaceutically acceptable carrier, diluent or excipient, for use in (i) eliciting a Type 1 interferon response, and/or (ii) enhancing the expression of one or more pro-inflammatory cytokines, and/or (iii) eliciting trained immunity, in a human subject infected by a virus, bacteria, protozoa or fungus, preferably wherein the virus is a coronavirus, and/or at elevated risk of exposure to and/or severity of said infection, wherein the non-pathogenic non-viable Mycobacterium is selected from M. vaccae, including the strain deposited under accession numbers NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. indicus pranii, M. w, M. manresensis, M. kyogaense (as deposited under DSM 107316/CECT 9546), M. tuberculosis Aoyama B or H37Rv, and combinations thereof, preferably the strain of Mycobacterium obuense deposited under the Budapest Treaty under accession number NCTC 13365.

In a ninth aspect of the invention, there is provided a method, adjuvant, pharmaceutical composition or use according to any preceding aspect, wherein the human subject at elevated risk of exposure to and/or severity of said viral infection, is at least 50, 55, 60, 65, 70, 75, 80, 85 or 90 or more years old.

In a tenth aspect of the invention, there is provided a method, adjuvant, pharmaceutical composition or use according to any preceding aspect, wherein the infection is selected from a respiratory tract infection, urinary tract infection, skin infection, or combinations thereof, optionally wherein a secondary bacterial or viral co-infection is prevented or reduced.

In an eleventh aspect of the invention, there is provided a method, adjuvant, pharmaceutical composition or use according to any preceding aspect, wherein said human subject at elevated risk of exposure to and/or severity of said infection demonstrates a reduction in immunosenescence following administration of said immunomodulator and one or more biologically-active agents, as exhibited by an upregulation of the cellular immune response such as the adaptive immune response, upregulation of the B cell, CD8+ T cell and CD4+ T cell response, and/or increase in T-cell repertoire and/or newly emerging thymic emigrants.

In a twelfth aspect of the invention, there is provided a method, adjuvant, pharmaceutical composition or use according to any preceding aspect, wherein said human subject at elevated risk of exposure to and/or severity of said infection demonstrates a reduction in inflammaging following administration of said immunomodulator and one or more biologically-active agents, as exhibited by a reduction in chronic (low grade) systemic inflammation, such as reduced levels of pro-inflammatory cytokines in peripheral blood, selected from: TNF-alpha or interleukin-6, TRAIL, CD38, CRP or serum amyloid alpha.

### DETAILED DESCRIPTION OF THE INVENTION

This invention is predicated on the surprising discovery that an immunomodulator comprising a non-pathogenic, non-viable Mycobacterium, such as Mycobacterium obuense, can be used in humans for the treatment and/or prevention of infections caused by a virus, bacteria, protozoa or fungus, in particular where the virus is a coronavirus, as well as the symptoms associated therefrom. Such coronaviruses may include Severe Acute Respiratory Syndrome associated Coronavirus (SARS-CoV), Middle East Respiratory Syndrome associated Coronavirus (MERS-CoV), and the SARS-CoV-2 virus discovered in late 2019, said ‘SARS-CoV-2” interchangeable with the terms ‘COVID-19’ or “COVID” throughout.

The Th1 type immune response plays a dominant role in an adaptive immunity to viral infections. Cytokine microenvironment generated by antigen presenting cells dictate the direction of T cell responses. Helper T cells orchestrate the overall adaptive response, while cytotoxic T cells are essential in killing of viral infected cells. Humoral immune response, especially production of neutralizing antibody, plays a protective role by limiting infection at later phase and prevents reinfection in the future. In SARS-CoV, both T and B cell epitopes have been extensively mapped for the structural proteins, S, N, M and E protein. SARS-CoV infection induces seroconversion as early as day 4 after onset of disease and was found in most patients by 14 days. Long lasting specific IgG and neutralizing antibody are reported as long as 2 years after infection. For MERS-CoV infection, seroconversion is seen at the second or third week of disease onset. For both types of coronavirus infections, delayed and weak antibody response are associated with severe outcome.

Limited serology details of SARS-CoV-2 have been reported. In a preliminary study, one patient showed peak specific IgM at day 9 after disease onset and the switching to IgG by the end of week two. Interestingly, sera from five patients of confirmed COVID-19 show some cross-reactivity with SARS-CoV, but not other coronavirus. Furthermore, all sera from patients were able to neutralize SARS-CoV-2 in an in vitro plaque assay, suggesting a possible successful mounting of the humoral responses.

There are early indications that antibodies, and especially neutralising antibodies, are not the predominant mechanism necessary for infected individuals to overcome a COVID-19 infection. Importantly, it was recently shown that a healthy patient with mild to moderate COVID-19 symptoms, who recovered quickly, had a broad-based robust immune response across different immune cell types, which was associated with clinical recovery [Thevarajan et al, 2020]. This observational study identified the presence of activated CD4+ T cells, CD8+T cells and follicular helper T cells. In addition, increased antibody-secreting cells (ASCs) and immunoglobulin M (IgM) and IgG antibodies were found in the blood of the patient, along with a higher frequency of CD38+ HLA-DR+CD8+ T cells relative to healthy individuals, which produced large amounts of granzymes A and B and perforin.

T cell response in SARS-CoV was extensively investigated. In one study using 128 convalescent samples, it was reported that CD8+ T cell responses were more frequent with greater magnitude than CD4+ T cell responses. Furthermore, the virus specific T cells from the severe group tended to be a central memory phenotype with a significantly higher frequency of polyfunctional CD4+ T cells (IFNγ, TNFα, and IL-2) and CD8+ T cells (IFNγ, TNFα and degranulated state), as compared with the mild-moderate group. Strong T cell responses correlated significantly with higher neutralizing antibody while more serum Th2 cytokines (IL-4, IL-5, IL-10) were detected in the fatal group. For the epitope mapping, most responses (70%) were found against the structural proteins (spike, envelope, membrane, and nucleocapsid). In MERS-CoV infection, early rise of CD8+ T cells correlates with disease severity and at the convalescent phase, dominant Th1 type helper T cells are observed. In an animal model, airway memory CD4+ T cells specific for conserved epitope are protective against lethal challenge and can cross react with SARS-CoV and MERS-CoV.

Clearly, current evidence strongly indicated that Th1 type response is key for successful control of SARS-CoV and MERS-CoV and also now true for SARS-CoV-2, as well.

Thus, in a first aspect of the invention, there is provided a method of treating or preventing an infection and/or the symptoms thereof in a human subject at elevated risk of exposure to and/or severity of said infection, said method comprising administering to the human subject an immunomodulator, wherein the immunomodulator comprises a non-pathogenic non-viable Mycobacterium, and wherein infection is caused by a virus, bacteria, protozoa or fungus, preferably wherein the virus is a coronavirus.

An immunomodulator, as defined according to the present invention, is a component which stimulates innate and type-1 immunity, including Th1 and macrophage activation and cytotoxic cell activity, as well as independently down-regulating inappropriate Th2 responses via immunoregulatory mechanisms.

In the present invention, the immunomodulator suitably comprises a non-pathogenic non-viable Mycobacterium obuense (commonly known as “IMM-101” and used interchangeably throughout). In preferred embodiments of the invention, the non-pathogenic non-viable Mycobacterium obuense is the strain deposited under the Budapest Treaty under accession number NCTC 13365.

SARS-CoV, MERS-CoV and SARS-CoV-2 are all highly pathogenic human coronaviruses responsible for acute and chronic diseases of the respiratory, hepatic, gastrointestinal and neurological systems. It is envisaged that the administration of a Mycobacteria, such as Mycobacterium obuense, may be used for the treatment and/or prevention of any infection caused by any virus, bacteria, protozoa or fungus, preferably wherein the virus is a coronavirus and the human subject is at elevated risk of exposure to and/or severity of said infection.

In a particular embodiment, the coronavirus may be SARS-CoV. In another embodiment, the coronavirus may be MERS-CoV. In a preferred embodiment, the coronavirus may be SARS-CoV-2.

Administration of mycobacteria, including M. obuense, induces a complex immune response in the host. Treatment with such a preparation will surprisingly stimulate innate and type-1 immunity, including Th1 and macrophage activation and cytotoxic cell activity. Administration of an immunomodulator comprising non-pathogenic non-viable M. obuense, for example, will also independently down-regulate inappropriate Th2 responses within a subject via immunoregulatory mechanisms that are each well characterised within the art. This would restore the healthy balance of the immune system, as would be appreciated by a person skilled in the art.

The demonstrated mechanism of action (MoA) of M. obuense and other such non-pathogenic, non-viable mycobacteria disclosed herein, is a multi-targeted, systemic immunomodulation of the innate and adaptive immune system, including but not limited to a rapid type 1 immune response with, systemic expansion of IFNγ producing activated NK cells, NKT-cells and γδ T-cells, as well as CD4+(Th1) and CD8+(perforin and granzyme producing CTL) T-cells. Such factors have been shown to be crucial for the human body to successfully overcome coronavirus infections. An analysis of T-cell repertoires in health care workers surviving SARS-CoV infections showed that effector memory Vγ9Vδ2 T-cell populations were selectively expanded at about 3 months after the onset of disease, without expansion of specific αβ T-cells (Poccia et al. 2006 J Infect Dis 1;193 (9): 1244-9). Expansion of the Vγ9Vδ2 T-cell population was associated with higher anti-SARS-CoV immunoglobulin G titres. In addition, in vitro experiments demonstrated that stimulated Vγ9Vδ2 T cells display an IFNγ-dependent anti-SARS-CoV activity, with the ability to directly kill SARS-CoV-infected target cells. Therefore, Vγ9Vδ2 T-cells may play a protective role during SARS or other similar infections by coronaviruses. An absolute majority of Vδ2 T-cells co-express Vγ9 (Poccia et al. 2006 J Infect Dis 1;193 (9): 1244-9). IMM-101's induced proliferation and upregulation of CD69, CD25 and HLA-DR on γδ T-cells is limited to the Vδ2 subset purported to be involved in the anti-SARS-CoV immunosurveillance in vivo. IMM-101 activated Vδ2 T-cells show enhanced effector responses, upregulated granzyme B expression, enhanced production of IFN-γ and TNF-α, and enhanced degranulation in response to tumour cells (Fowler et al. 2012 Cancer Immunol Immunother 61: 535-547).

IMM-101 (Mycobacterium obuense) in particular, has been shown to be well tolerated in over 300 patients with advanced cancer in clinical trials and compassionate use programs. IMM-101 demonstrated further clinical benefit in a randomized Phase 2 study in advanced pancreatic cancer (Dalgeish et al. 2016 BR J Cancer 115(7): 789-796).

It is envisaged that the immunomodulator IMM-101 may be used in prophylaxis of populations in general but also those at high risk of coronavirus infection, including health care workers, cancer patients, and persons who have been in close contact to infected patients, as well as the elderly. Such prophylaxis may be achieved by changing the immune surveillance status and disease trajectory and/or severity. It is also envisaged that the immunomodulator of the present invention may be used as a co-treatment of already infected patients. The term “non-pathogenic” herein refers to the characteristic of the Mycobacteria of the present invention in being unable to cause disease, harm or death to the human host of which they are administered.

By “non-viable”, it is meant that the Mycobacterium have been microbiologically inactivated through certain means of cell-killing. Methods to enable or enforce such non-viability may include heat-killing, extended freeze-drying (Tolerys SA), irradiation by gamma waves or electron beam, or subjecting the mycobacteria to chemicals such as formaldehyde. Such preparation during manufacture would mean the organism is not associated with side-effects known from delivering live or attenuated organisms.

By “trained immunity” is meant the ability of one antigenic stimulus to elicit more potent immune responses to a second, different antigen administered at a later time. Trained immunity is antigen-independent, based on innate immune cell activation and heterologous CD4 and CD8 memory activation, cytokine, chemokine or other soluble or membrane bound molecule mediated, and is associated with epigenetic and metabolic changes.

In a preferred embodiment of the invention, the human subject at elevated risk of exposure to and/or severity of said infection is immunocompromised. Accordingly, the human subject may have congenital or acquired immune deficiencies, selected from, (i) concurrent disease such as vitamin D deficiency, AIDS, leukemia, lymphoma and other cancers, (ii) cancer therapy such as exposure to cytotoxic drugs, radiation, (iii) immunosuppressive therapy such as corticosteroids, or (iv), age-related impaired immune function.

In another preferred embodiment of the invention, the human subject at elevated risk of exposure to and/or severity of said infection has one or more comorbid conditions.

In certain embodiments, the one or more comorbid conditions are selected from metabolic disorder, obesity, diabetes, asthma, COPD, hypertension, cardiac disease, arrhythmia, renal disease, liver disease, hematolgic disease, dementia and other neurological disorder, rheumatolgical disease, malnutrition, thrombosis or cancer, optionally wherein said comorbidity is chronic. The cancer may accordingly be non-immunocompromising.

In yet another preferred embodiment of the invention, the human subject at elevated risk of exposure to and/or severity of said infection is, (i) a member of a household where one or more occupants has known or suspected infection, such as a viral infection associated with a coronavirus, or (ii) a healthcare or social care worker exposed to human subjects with known or suspected infection, such as a viral infection associated with a coronavirus.

In a further embodiment of the invention, the infection is selected from a respiratory tract infection, urinary tract infection, skin infection, or combinations thereof, optionally wherein a secondary bacterial or viral co-infection is prevented or reduced. The respiratory tract infection may suitably be an upper respiratory tract infection (uRTI) and may be acute, chronic or recurrent. The urinary tract may similarly be acute, chronic or recurrent. Furthermore, the infection may be febrile or non-febrile, mild or severe.

In another embodiment of the invention, the infection is associated with a herpes zoster (cold sore) infection, whereby it is an acute or recurrent infection and where the immunomodulator prevents or reduces said herpes zoster infection or re-infection.

In a further aspect of the invention, there is provided a method of enhancing the immunogenicity and response to a subsequent vaccination in a human subject at elevated risk of exposure to and/or severity of infection, said method comprising administering to the human subject an immunomodulator, wherein the immunomodulator comprises a non-pathogenic non-viable Mycobacterium, and wherein said vaccination is selected from: influenza (inactivated, live attenuated or recombinant); tetanus, diphtheria, pertussis (Tdap ort Td); measles, mumps rubella, (MMR); Varicella; Zoster (live or recombinant); human papillomavirus (HPV); pneumococcal conjugate (PCV13); pneumococcal polysaccharide (PPSV23); hepatitis A; hepatitis B; meningococcal ACWY; meningococcal B; Haemophilus influenza type B. Other suitable vaccines to be boosted include travel vaccines such as typhoid, paratyphoid, Yellow Fever, rabies and Japanese Encephalitis.

In preferred embodiments of the invention, the non-pathogenic, non-viable Mycobacterium is the rough variant, preferably the rough variant of M. obuense.

In preferred embodiments of the invention, the non-pathogenic, non-viable Mycobacterium, suitably Mycobacterium obuense, is in a substantially whole cell form, such as where more than 50% or more of the mycobacteria in suspension are greater than 1 to 10 microns in diameter, as measured by laser diffraction (e.g. D50 value), or is in a form which has not been exposed to high pressure processing or other conditions to induce substantial cell lysis.

As would be understood by the skilled person, rough variants of M. obuense, for example, would lack cell surface-associated glycopeptidolipids (GPL) resulting in a characterised rough morphology with non-motile and non-biofilm-forming properties, as described in Roux et al. 2016, Open Biol 6: 160185. The amount of Mycobacterium administered to the patient in the present invention would be sufficient to elicit a protective immune response in the patient such that the patient's immune system would be able to mount an effective immune response against the infecting agent, suitably a coronavirus.

In certain embodiments of the invention, the amount of non-pathogenic non-viable Mycobacterium, such as M. obuense, administered to the subject may be from 10³ to 10¹¹ organisms, preferably from 10⁴ to 10¹⁰ organisms, and more preferably from 10⁶ to 10¹⁰ organisms per unit dose. In a most preferred embodiment, the effective amount of non-pathogenic non-viable Mycobacterium administered, suitably M. obuense, is from 10⁷ to 10⁹ organisms per unit dose.

In some embodiments, the amount of non-pathogenic non-viable Mycobacterium administered may be from 0.0001 mg to 1 mg per unit dose, suitably M. obuense. In a preferred embodiment, the amount of non-pathogenic non-viable Mycobacterium administered may be between 0.1 mg and 1 mg per unit dose, optionally wherein the unit dose is administered on two or more separate occasions.

In another embodiment, the human subject is administered an intradermal priming or initial dose of preferably at least about 0.5 mg Mycobacterium per unit dose, such as about 1.0 mg, and one or more subsequent or boosting intradermal doses of not more than about 0.5 mg Mycobacterium per unit dose, suitably where the Mycobacterium is M. obuense.

In certain embodiments of the invention, the amount of non-pathogenic non-viable Mycobacterium administered is between 0.0001 mg and 1 mg per unit dose, optionally wherein the unit dose is administered on two or more separate occasions separated by at least 7 days or more, such as administration on each of day 0, day 14 (+/−1, 3 or 5 days or more), and optionally day 30 (+/−5, 7 or 10 days or more) or day 45 (+/−7, 10 or 14 days or more), suitably wherein the Mycobacterium is M. obuense.

In some embodiments, the amount of non-pathogenic non-viable Mycobacterium administered may be from 0.0001 mg to 1 mg per dose wherein the dose is administered 1, 2, 3, 4, 5, 6, 10 or 20 or more times over a number of days, weeks, or months, suitably wherein the Mycobacterium is M. obuense.

In other embodiments of the invention, the amount of non-pathogenic non-viable Mycobacterium administered may be from 0.0001 mg to 1 mg per dose, wherein the dose initially comprises two injections of 0.1 mg to 0.5 mg in each deltoid, or two injections of 1.0 mg in each deltoid, followed by a second dose of either 0.1, 0.2, 0.5 or 1.0 mg 7 or 14 days or more later, optionally with further doses administered over the subsequent weeks or months until resolution of said infection and or said symptoms.

Alternatively, the amount of non-pathogenic non-viable Mycobacterium administered may be from 0.0001 mg to 1 mg or more per dose, administered three times per day for three consecutive days, suitably for the treatment of severe or critical SARS-Cov-2 infections.

It is envisaged that the immunomodulator of the present invention may be prepared and/or purified in the form of various fractions common in the art of administration, including but not limited to lysates, homogenates, and sonicates of the Mycobacterium

The non-pathogenic non-viable Mycobacterium, such as M. obuense, may be administered to the patient via the parenteral, oral, sublingual, nasal or pulmonary route. In a preferred embodiment, the immunomodulator is administered via a parenteral route selected from subcutaneous, intradermal, subdermal, intraperitoneal, or intravenous injection. In a most preferred embodiment, administration by the parenteral route may comprise intradermal injection of M. obuense.

Further preferably, the parenteral route is selected from subcutaneous, intradermal, subdermal, intraperitoneal, intravenous, peritumoral, perilesional, intralesional or intratumoral, and combinations thereof, optionally wherein said subject has one or more tumours. Suitably, intratumoral administration may be sequentially followed by intradermal administration.

In some embodiments the non-pathogenic non-viable Mycobacterium, such as M. obuense, may be administered in an intradermal injection via a microneedle device comprising a plurality of needles. In another embodiment, the plurality of microneedles may be deployed in a line, square, circle, grid, or array. In other embodiments, the microneedle device may include between 2 and 5000 microneedles per square centimetre, such as between 4 and 1500 microneedles per square centimetre, of between 10 and 1000 microneedles per square centimetre or between 2 and 500 microneedles per square centimetre. In a further embodiment, the microneedles may be between 2 and 2000 microns in length, such as between 20 and 1000 microns, or between 50 and 500 microns, or between 100 and 400 microns. Suitable microneedle devices include: North Carolina State University (as described in WO2017/151727), Debioject microneedle (Debiotech, Switzerland), Micronject600 (NanoPass, Israel, as described in WO2008/047359), Nanopatch (Vaxxas, USA), SOFUSA (Kimberly-Clark, USA, as described in WO2017/189259 and WO2017/189258), Micron Biomedical's dissolving microarray, and the MIMIX dissolving, controlled release microarray (Vaxess, USA), plus 3M's Microstructured Transdermal Systems (MTS) or Zosano's titanium microprojection array employed in Qytpta (zolmitriptan intracutaneous microneedle system) or Macroflux.

In some embodiments, the microneedles are solid. In other embodiments, the microneedles are hollow. In a further embodiment, the microneedles may be configured to deliver the non-pathogenic non-viable Mycobacterium, such as M. obuense, intradermally, optionally wherein said Mycobacterium is delivered to the lymphatic vessels. In yet a further embodiment, the non-pathogenic non-viable Mycobacterium, may be coated onto or embedded within at least a portion of the microneedles, optionally wherein the microneedles are implanted into or removable from the skin. Preferably, said coating or microneedle would be dissolvable upon contact with the skin.

In some embodiments of the invention, the immunomodulator comprises non-pathogenic non-viable Mycobacterium, such as M. obuense, and may be administered via a single-use pre-filled syringe. In other embodiments, the immunomodulator may be administered via an automated, multi-use, or disposable cartridge jet injector, such as the Tropis ID' (WHO approved for polio) and ‘Stratis’ device, which is FDA approved for flu (both ex Pharmajet).

In some embodiments of the present invention, the immunomodulator comprising non-pathogenic non-viable Mycobacterium, such as M. obuense, may be administered for the treatment or prevention of infections caused by a coronavirus in combination with one or more other antiviral therapies. Such antiviral therapies may include administration of oseltamivir phosphate (Tamiflu®), zanamivir (Relenza®), peramivir (Rapivab®), baloxavir marboxil (Xofluza®), Remdesivir lopinavir/ritonavir (Kaletra/Aluvia®). Such antiviral therapies may be administered simultaneously, separately or sequentially with the non-pathogenic non-viable Mycobacterium, such as M. obuense.

In a further embodiment, the antiviral therapy is administered via the same or different route of administration as the non-pathogenic non-viable Mycobacterium, for example, the non-pathogenic non-viable Mycobacterium, suitably M. obuense, and antiviral agent are each administered via nasal or oral inhalation.

The phrase “pharmaceutically acceptable” refers to molecular entities and compositions that do not produce an adverse, allergic or other untoward reaction when administered to a human, as appropriate. The preparation of a pharmaceutical composition that contains mycobacteria will be known to those of skill in the art in light of the present disclosure, as exemplified by Remington's Pharmaceutical Sciences, 18th Ed. Mack Printing Company, 1990, Moreover, for human administration, it will be understood that preparations should meet sterility, pyrogenicity, general safety and purity standards. A specific example of a pharmacologically acceptable carrier as described herein is borate buffer or sterile saline solution (0.9% NaCl).

As used herein, “pharmaceutically acceptable carrier” includes any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18th Ed. Mack Printing Company, 1990, pp. 1289-1329).

In preferred embodiments of the invention, the non-pathogenic non-viable Mycobacterium is selected from M. vaccae, including the strain deposited under accession number NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. indicus pranii, M.w, M. kyogaense (as deposited under DSM 107316/CECT 9546), M. manresensis, M. tuberculosis Aoyama B or H37Rv, RUTI, z-100 and combinations thereof, preferably the strain of Mycobacterium obuense deposited under the Budapest Treaty under accession number NCTC 13365.

In other embodiments of the invention, the non-pathogenic non-viable Mycobacterium is the rough variant and/or whole cell, preferably the rough strain of Mycobacterium obuense deposited under the Budapest Treaty under accession number NCTC 13365.

In another embodiment of the invention, the non-pathogenic non-viable Mycobacterium may include BCG in heat-killed, extended freeze-dried, irradiated or chemically-killed form.

In another embodiment of the invention, the non-pathogenic non-viable Mycobacterium does not include BCG in live, attenuated form.

In preferred embodiments of the invention, the one or more biologically-active agent is a therapeutic drug, nutraceutical, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof.

In one embodiment of the invention, the nutraceutical is selected from amino acids, antioxidants, fats, vitamins, trace elements, minerals, micronutrients, plant extracts, phytochemicals, fibres, prebiotics, probiotics, and/or a combination thereof, preferably vitamin D, vitamin C and/or zinc. Vitamin D may have a pleiotropic effect in immune cells, including macrophages, and an immunomodulatory role of vitamin D in various immune cells and diseases has been demonstrated. Accordingly, suitable presentations of vitamin D may include oral or injected formulations of paracalcitol e.g. 1 mcg orally; cholecalciferol, 60,000 IU orally per week or single injections of 300,000 IUs; or 300,000 IUs of ergocacliferol injected.

In another embodiment of the invention, the one or more biologically-active agent is selected from an antiviral, such as favipiravir, remdesivir, danoprevir, ritonavir, triazavirin, umifenovir, darunavir; aviptadil; an anti-viropin; an furan inhibitor; convalescent sera; azvudine; leronlimab; remestemcel-L; TJM2 (TJ003234); Brilacidin; rintatolimod; sarilumab; piclidenoson; tocilizumab and other anti-IL-6 antibodies; anti-PD1 or anti-PD-L1 mabs; tranilast; TD-0903 (optionally via inhalation); GD-31; Stem Cell Therapy for Pneumonia; jaktinib hydrochloride; lenzilumab; brilacidin; efineptakin alfa; opaganib; Panaphix; upamostat; CYNK-001; IFX-1; PAX-1; ChAdOx1 nCoV-19; CMAB-806; mRNA-1273; TAK-888; Stem Cell Therapy for Coronavirus Disease 2019 (COVID-19); BDB-001; Giapreza; angiotensin II inhibitors; hydroxychloroquine; chloroquine; Famotidine or its Polymorphs; NK cells; Immunoglobulin from cured patients; Ankylosaurus; aerosol inhalation of vMIP (viral macrophage inflammatory protein); dihydroartemisin; carriomycin; suramin sodium; fingolimod; leflunomide; thalidomide; kinase inhibitors such as ruxolitinib; BTK inhibitors such as Acalabrutinib (Calguence); Adalimumab; camrelizumab; eculizumab; acetylcysteine; ACE inhibitors; angiotensin receptor blockers; Diammonium glycyrrhizinate; Dipyridamole; ebastine; inhaled gases such as oxygen, hydrogen, nitric oxide, and combinations thereof; pirfenidone; rhG-CSF; LMW heparin, optionally low-dose; enoxaparin; Recombinant Protein VSF for Viral Infections; ONCase-PEG; (ASC-09+ritonavir); Recombinant Protein for Pneumonia; pritumumab; APN-01; mifamurtide; pentoxifylline; tacrolimus; paclitaxel; sorafenib; ciclesonide; dornase alpha; dexamethasone; ifenprodil; INO-4800; Oligonucleotide for Coronavirus Disease 2019 (COVID-19); OYA-1; Recombinant Protein to Agonize GCSFR for Pneumonia; TNX-1800; AT-100; BPI-002; El DD-2801; TZLS-501; Antisense RNAi Oligonucleotides for Coronavirus Disease 2019 (COVID-19); Fusion Protein for Coronavirus Disease 2019 (COVID-19); Gene Therapy for Coronavirus Disease 2019 (COVID-19); LUNAR-COV19; Protein subunit vaccine (Matrix-M) and Monoclonal Antibodies for Coronavirus Disease 2019 (COVID-19).

In other embodiments of the invention, the biologically-active agent is an anti-CD38 mab (for example daratumumab, isatuximab, MOR202 and TAK-079), an A2R antagonist, an adenosine receptor 2 antagonist; SCH 58261, A2a adenosine receptor antagonist; PSB 1115, A2b adenosine receptor antagonist, optionally an anti-PD-1 or anti-PD-L1 antibody.

In other embodiments of the invention, the biologically-active agent is an interferon, such as interferon alpha, beta or gamma, suitably interferon alpha 1b or alpha 2b, novaferon, wherein said interferon is preferably administered by injection, nasal drops or spray, or inhalation via a nebulizer and such like, suitably the preparation called SNG001 (inhaled interferon beta la from Synairgen, UK). The interferon may be administered at the same time and/or via the same route as the Mycobacterium, or at separate times and/or via separate routes of administration.

In other embodiments of the invention, the biologically-active agent is thymosin or a purified fraction thereof, such as thymosin alpha or thymalfasin (Zadaxin), optionally injected once per week or delivered nasally or via oral aerosol inhalation. The thymosin or thymalfasin may be administered at the same time and/or via the same route as the Mycobacterium, or at separate times and/or via separate routes of administration.

In other embodiments of the invention, the biologically-active agent is niclosamide, optionally injected once per week, orally administered in tablet/capsule form, or delivered nasally or via oral aerosol inhalation. The niclosamide may be administered at the same time and/or via the same route as the Mycobacterium, or at separate times and/or via separate routes of administration.

In another embodiment of the invention, the biologically-active agent is the anti-cancer drug lenalidomide; it is one of the very few pleiotropic agents that not only lowers the expression of TNF-α, IL-6, and IL-8, but also increases the expression of anti-inflammatory cytokines (e.g., IL-10). Thus, lenalidomide modulates both innate and adaptive immune responses.

In other embodiments of the invention, the biologically-active agent is a geroprotector selected from mTOR inhibitors, such as rapamycin, everolimus or metformin.

In another embodiment of the invention, the biologically-active agent is a fluoroquinolone antibiotic, preferably levofloxacin, administered orally or via aerosol inhalation. It has been reported that fluoroquinolones also function as an immunomodulator, an anti-oxidant agent and a nitric oxide (NO) regulator. Thus, they may have the potential to inhibit influenza virus-induced pneumonia via its pleiotropic effects, including its anti-oxidative and NO inhibitory properties.

In a further embodiment, administration of the immunomodulator according to the invention, leads to activation and maturation of dendritic cells (DC) and prevents the strong induction of TNF-related apoptosis-inducing ligand (TRAIL) gene expression in coronavirus infected DCs and/or other antigen presenting cells, postulated to be responsible for lymphoid depletion in SARS-Cov2 patients. Administration may also lead to induction of G-CSF, IL-3 and IL-6 expression and drive subsequent emergency granulopoeisis and an increase in neutrophils resulting in protection from sepsis.

In other embodiments of the invention, the biologically-active agent is an antigen or antigenic determinant.

In further embodiments of the invention, the antigen or antigenic determinant is specific for, targeted to and/or derived from a coronavirus, or is a vehicle used for delivery thereof, such as an adenoviral vector, vaccinia vector, plasmid vector, optionally live attenuated; mRNA, a modified mRNA, or a stabilized mRNA; viral replicase, spike protein, spike fragment, envelope protein, membrane protein, nucleocapsid protein, subunit of a spike protein, receptor binding domain (RBD) of the subunit of a spike protein, or a functional fragment or variant thereof.

Suitably, the antigen may even be engendered within the host tissues as part of a disease process. The antigen may originate from a viral, bacterial, host or parasitic invasion, or may be a substance released from the tissues such as a stress protein or a killed coronavirus.

In preferred embodiments of the invention, the immunomodulator and/or one or more biologically-active agents are administered via a parental, oral, sublingual, nasal or pulmonary route.

In further preferred embodiments of the invention, the immunomodulator and/or one or more biologically-active agents are administered in the same composition via the same route or in separate compositions each via a different route, optionally at the same time or different times.

In further embodiments of the invention, the parental route is selected from subcutaneous, intradermal, subdermal, intraperitoneal or intravenous injection, preferably wherein the parental route is an intradermal injection.

For example, the immunomodulator and one or more biologically-active agents are both administered nasally, the immunomodulator is administered intradermally and one or more biologically-active agents are administered nasally.

In another embodiment of the invention, the immunomodulator and/or one or more biologically-active agents are administered in an intradermal injection via a microneedle device comprising a plurality of needles, optionally wherein said microneedles are hollow.

In another embodiment of the invention, the immunomodulator and/or one or more biologically-active agents are prepared or presented for administration within a single-use pre-filled syringe or multi-dose applicator or jet injector, or nasal delivery device or oral inhaler.

In a further preferred embodiment of the invention, the adjuvant is for use in conjunction with one or more biologically-active agents wherein said one or more biologically-active agent is a therapeutic drug, nutraceutical, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof.

In a further preferred embodiment of the invention, the adjuvant is for use in conjunction with an antigen or antigenic determinant.

In a further embodiment of the invention, the adjuvant is for use in conjunction with an antigen or antigenic determinant which is specific for, targeted to and/or derived from a coronavirus, such as an adenoviral vector, vaccinia vector, plasmid vector, optionally live attenuated; mRNA, a modified mRNA, or a stabilized mRNA; viral replicase, spike protein, spike fragment, envelope protein, membrane protein, nucleocapsid protein, subunit of a spike protein, receptor binding domain (RBD) of the subunit of a spike protein, or a functional fragment or variant thereof.

In a further embodiment of the invention, the adjuvant is for use in conjunction with an antigen or antigenic determinant which is specific for, targeted to and/or derived from a coronavirus selected from SARS-CoV-2, SARS-CoV and MERS-CoV.

In a further embodiment of the invention, the adjuvant comprises a non-pathogenic non-viable Mycobacterium selected from M. vaccae, M. obuense, M. parafortuitum, M. aurum, M. indicus pranii, M. w, M. manresensis, z-100, RUTI and combinations thereof, preferably the strain of Mycobacterium obuense deposited under the Budapest Treaty under accession number NCTC 13365.

In a further embodiment of the invention, the adjuvant comprises a non-pathogenic non-viable Mycobacterium and is a rough variant and/or presented in substantially in whole cell form.

In a preferred embodiment of the invention, the adjuvant comprises a non-pathogenic non-viable Mycobacterium and is administered via a parenteral, oral, sublingual, nasal or pulmonary route.

In a preferred embodiment of the invention, the adjuvant comprising a non-pathogenic non-viable Mycobacterium and/or one or more biologically-active agents are administered in the same composition via the same route or in separate compositions each via a different route, optionally at the same time or different times.

In a further embodiment of the invention, the adjuvant comprises a non-pathogenic non-viable Mycobacterium and/or the one or more biologically-active agents are each administered via a parenteral route selected from subcutaneous, intradermal, subdermal, intraperitoneal or intravenous injection, suitably wherein the parenteral route is an intradermal injection.

In yet a further embodiment of the invention, the adjuvant comprising a non-pathogenic non-viable Mycobacterium and/or one or more biologically-active agents are administered in an intradermal injection via a microneedle device comprising a plurality of needles, optionally wherein said microneedles are hollow.

In yet a further embodiment of the invention, the adjuvant comprising a non-pathogenic non-viable Mycobacterium and/or one or more biologically-active agents are prepared for administration within a single-use pre-filled syringe or multi-dose applicator or jet injector.

In another embodiment of the invention, the adjuvant comprises a non-pathogenic non-viable Mycobacterium and where the amount of non-pathogenic non-viable Mycobacterium administered is from 10⁴ to 10⁹ cells per unit dose, or is between 0.0001 mg and 1 mg per unit dose.

In yet a further embodiment of the invention, the adjuvant comprising a non-pathogenic non-viable Mycobacterium modifies a cellular immune response.

In yet a further embodiment of the invention, the adjuvant comprises a non-pathogenic non-viable Mycobacterium which is administered for the treatment and/or prevention of an infection caused by a coronavirus, optionally with one or more biologically-active agents, by; (i), eliciting a Type 1 interferon response, and/or; (ii), enhancing the expression of one or more pro-inflammatory cytokines, and/or; (iii), eliciting trained immunity in the human subject, optionally wherein the adjuvant comprises the non-pathogenic non-viable Mycobacterium obuense strain deposited under the Budapest Treaty under accession number NCTC 13365.

In yet a further embodiment of the invention, the adjuvant or method according to the invention induces trained immunity, which is demonstrated by (i) increasing the non-specific effector response of innate immune cells to pathogens, including monocyte/macrophages; and/or (ii), increasing adaptive T cell responses to both specific and non-related (bystander) antigens; and/or (iii), an increase in heterologous CD4 and CD8 memory activation; and/or (iv), epigenetic and metabolic changes, such as histone methylation, and/or (v), an upregulation of IL-1 beta.

In yet a further embodiment of the invention, is the use of an adjuvant or pharmaceutical composition according to the invention disclosed herein, in the manufacture of a medicament for the treatment or prevention of a viral infection and/or the symptoms thereof in a subject, wherein said viral infection is caused by a coronavirus selected from SARS-CoV-2, SARS-CoV and MERS-CoV.

In yet a further embodiment of the invention, is the use of an adjuvant or pharmaceutical composition or method according to the invention disclosed herein, in the manufacture of a medicament comprising a non-pathogenic non-viable Mycobacterium, for the treatment or prevention of a viral infection and/or reduction in the incidence, severity and/or onset of symptoms thereof in a human subject at elevated risk of exposure to and/or severity of said viral infection, wherein said symptoms include: respiratory (cough, sputum, sore throat, runny nose, ear pain, wheeze, shortness of breath, and chest pain); systemic (fever, confusion, myalgia, joint pain, skin ulcers, rash, seizures, strokes, infarctions, embolisms, lymphadenopathy and fatigue); enteric (abdominal pain, vomiting and diarrhoea), wherein the non-pathogenic non-viable Mycobacterium is selected from M. vaccae, M. obuense, M. parafortuitum, M. paragordonae (strain 49061), M. aurum, M. indicus pranii, M. w, M. kyogaense (as deposited under DSM 107316/CECT 9546), M. manresensis, M. tuberculosis Aoyama B or H37Rv, RUTI, z-100 and combinations thereof, preferably the strain of Mycobacterium obuense deposited under the Budapest Treaty under accession number NCTC 13365.

In yet a further embodiment of the invention, is the use of an adjuvant or pharmaceutical composition or method according to the invention disclosed herein, in the manufacture of a medicament comprising a non-pathogenic non-viable Mycobacterium, for the treatment or prevention of a viral infection in a human subject at elevated risk of exposure to and/or severity of said viral infection and/or infected with said virus, as demonstrated by a reduction of one or more of the following endpoints within 30, 60 or 180 days or more of first administering said non-pathogenic non-viable Mycobacterium: number of days of unplanned absenteeism for any reason; the cumulative incidence of documented SARS-CoV-2 infection; the number of days of unplanned absenteeism, because of documented SARS-CoV-2 infection; the number of days of absenteeism, because of imposed quarantine as a result of exposure to SARS-CoV-2 infection; the number of days of absenteeism, because of imposed quarantine as a result of having acute respiratory symptoms, fever or documented SARS-CoV-2 infection; the number of days of unplanned absenteeism because of self-reported acute respiratory symptoms; the number of days of self-reported fever (≥38 C); the number of days of self-reported acute respiratory symptoms; the cumulative incidence of self-reported acute respiratory symptoms; the cumulative incidence of death for any reason; the cumulative incidence of death due to documented SARS-CoV-2 infection; the cumulative incidence of Intensive Care Admission for any reason; the cumulative incidence of Intensive Care Admission due to documented SARS-CoV-2 infection; the cumulative incidence of Hospital Admission for any reason; the cumulative incidence of Hospital Admission due to documented SARS-CoV-2 infection; the incidence and magnitude of SARS-CoV-2 antibodies.

In yet a further embodiment of the invention, is the use of an adjuvant or pharmaceutical composition or method according to the invention disclosed herein, including the manufacture of a medicament comprising a non-pathogenic non-viable Mycobacterium, for the treatment or prevention of a viral infection in a human subject at elevated risk of exposure to and/or severity of said viral infection and/or infected with said virus, wherein said disease trajectory and/or severity is suppressed and/or reduced. Disease severity calculated using the Covid Severity Scale Scoring of 0-10. A score of 10 is worse and a score of 0 is best. Disease severity score will be based on the level of care required for individuals who test positive for COVID19 as follows: non-hospital-based care; patient hospitalized but no oxygen required; hospitalized and oxygen required; patient treated in intensive care and/or on mechanical ventilation; patient died. The ROX index, defined as the ratio of oxygen saturation as measured by pulse oximetry/F102 to respiratory rate, has been assessed as a predictor of the need to intubate and is, accordingly, improved by the invention disclosed herein.

Additional WHO criteria for severity include severe pneumonia, respiratory failure, acute respiratory distress syndrome (ARDS), sepsis and septic shock. Accordingly, the non-pathogenic non-viable Mycobacterium may prevent or reduce the incidence or severity of pneumonia, respiratory failure, acute respiratory distress syndrome (ARDS), sepsis and septic shock.

Early pathological findings of COVID-19 patients with ARDS, showed not only reduced counts of peripheral CD4 and CD8 T cells, but T cells were found in a hyperactivated state with high proportions of HLA-DR and CD38+ double-positive fractions. In another recent study in COVID-19 patients it has been shown that considerable proportions of peripheral CD4+ and CD8+ T cells co-expressed CD38 and HLA-DR, but those cells could not be re-activated with peptide pools of the S protein in vitro. Supporting the notion of SARCS-CoV-2 specific refractory T cells. It is known form Infection with HIV-1 induces lymphoid activation, resulting in an increase in T cell activation—associated antigens, such as CD38. Several studies have shown that such increased CD8+CD38+ T cell expression is a strong predictive marker for disease progression in HIV-1 infection. The CD8+CD38+ T cell count, not only predicts progression of HIV disease to AIDS and death, but it also offers additional independent predictive value for evaluation of plasma virus load (VL) and CD4+ T cell count.

The invention also encompasses the use of CD38+HLA-DR+PD-1+CD8+ T cells as measured over a prolonged period of time such as 30, 40, 50 or 60 days or more, as a prognostic indicator of SARS-Cov-2 disease severity.

In a further preferred embodiment of the invention, there is a reduction in; (i) the incidence of symptomatic and asymptomatic COVID-19 disease determined by RT-PCR and serology testing, (ii) the incidence of severe COVID-19 infection resulted in hospitalization and/or admission to intensive care units, (severity of COVID-19 disease), (iv) the incidence, rate and severity of febrile or non-febrile respiratory disease, and/or (iv) absenteeism (days off work), as measured over the 1, 3, 6 or 12 months following first administration of a medicament comprising a non-pathogenic non-viable Mycobacterium, wherein said human subject at elevated risk of exposure to and/or severity of said infection is a healthcare worker or social care worker exposed to SAR-CoV-2, or a cancer patient.

In addition, the non-pathogenic non-viable Mycobacterium prolongs the time to first SAR-CoV-2 proven respiratory illness or clinical features and reduces the incidence and/or severity of symptoms, including ‘flu-like’ symptoms such as fever, cough, and shortness of breath or less common signs and symptoms of COVID-19 infection including headache, muscle pain, abdominal pain, diarrhea, sputum production, anosmia, stroke, and sore throat.

In a further preferred embodiment of the invention, the non-pathogenic non-viable

Mycobacterium reduces the rate of “flu-like illness” which includes WHO definition of ILI [Fitzner 2018] or confirmed viral/bacterial respiratory infection.

In a further preferred embodiment of the invention, the non-pathogenic non-viable Mycobacterium administration, when compared with control patients (observation), results in no significant change or delay in cancer treatment or requirement for an unscheduled medical assessment (i.e. emergency room visit, family physician assessment, etc.), hospitalization, or death, in subjects suffering from cancer.

In another embodiment of the invention, incidence of COVID-19 seroconverted patients between baseline, 3 months, 6 months and 12 months is reduced.

In a further embodiment of the invention, rate of severe COVID-19 infection defined as a confirmed COVID-19 infection leading to hospitalization, ICU admission or death, is reduced.

Furthermore, compared to placebo, the invention herein reduces severity of COVID-19 disease (Outcome) and/or reduces the incidence and severity of febrile or afebrile respiratory disease measured over 6 months or more, in healthcare workers exposed to SARS-CoV-2 and/or cancer patients.

The invention herein reduces the incidence and or severity of recurrent herpes simplex recurrences in high risk subjects, preferably heath care workers, social care workers or cancer patients.

The invention may further may reduce time-to-first clinical worsening event consisting of any of the following: hospitalization for COVID-19 infection, Intensive care unit admission for COVID-19 infection, ventilation for COVID-19 infection or all cause death.

The invention herein reduces incidence, frequency, and severity of AEs considered possibly, probably or definitely related to receipt of said non-pathogenic non-viable Mycobacterium.

In a further preferred embodiment of the invention, administration of the non-pathogenic non-viable Mycobacterium increases failure-free survival (FFS), as defined as time from enrolment to recurrence or progression declared by the investigator on the basis of objective standard evaluation consistent with the disease site (e.g. cross-sectional imaging, serum tumour markers, etc.) or death.

In another embodiment of the invention, is the use of an adjuvant or pharmaceutical composition or method according to the invention disclosed herein, in the manufacture of a medicament comprising a non-pathogenic non-viable Mycobacterium, for the treatment or prevention of a viral infection in a human subject at elevated risk of exposure to and/or severity of said infection and/or infected with said virus, suitably a coronavirus, as demonstrated by a reduction in D-dimer levels, lymphopenia, elevated aminotransaminase levels, elevated lactate dehydrogenase levels, improvement of the neutrophil-to-lymphocyte ratio (NLR) and elevated inflammatory markers.

Lymphopenia is especially common in COVID-19 patients, even though the total white blood cell count can vary. For example, in a series of 393 adult patients hospitalized with COVID-19 in New York City, 90 percent had a lymphocyte count <1500/microL; leukocytosis (>10,000/microL) and leukopenia (<4000/microL) were each reported in approximately 15 percent. Common laboratory findings among hospitalized patients with COVID-19 include lymphopenia, elevated aminotransaminase levels, elevated lactate dehydrogenase levels, and elevated inflammatory markers (e.g., ferritin, C-reactive protein (CRP), and erythrocyte sedimentation rate).

In yet a further preferred embodiment of the invention, is the use, method, adjuvant or pharmaceutical composition according to the invention, wherein the medicament or pharmaceutical composition is a vaccine.

In a ninth aspect of the invention, there is provided a method, adjuvant, pharmaceutical composition or use according to the invention disclosed herein, wherein the human subject is at least 50, 55, 60, 65, 70, 75, 80, 85 or 90 or more years old.

In yet a further preferred embodiment of the invention is a method, adjuvant, pharmaceutical composition or use according to the invention, wherein said human subject demonstrates a reduction in immunosenescence, as exhibited by an upregulation of the cellular immune response and/or increase in T-cell repertoire.

In a further embodiment of the invention, there is provided a method, adjuvant, pharmaceutical composition or use according to the disclosure herein, wherein a secondary bacterial or viral co-infection is prevented or reduced.

TLR4 is known to sense lipopolysaccharide (LPS) from gram-negative bacteria but based on its additional function as sensor for damage-associated molecular patterns (DAMPs), TLR4 has been suggested to play a central roles in the induction of damaging inflammatory responses during several acute viral infections. In addition, oxidized phospholipids (OxPLs), shown to be leading to ALI in patients infected with SARS-CoV, accumulate in lungs of patients infected with SARS-CoV-2 as well and activate monocyte-derived macrophages through TLR4. Interfering with monocyte and endothelial cell activation by TLR4 in response to OxPLs may therefore help prevent thrombotic complications, recently identified and major factor in mortality of COVID-19 patients. The effects of IMM-101 (M. obuense suspension) are in part mediated by TLR2/1 and to a lesser extent TLR2/6 and other PPRs. TLR2 has been shown to directly trigger Th1 effector functions in mice. Subsequently, it was shown that IMM-101 activates human Mincle reporter cell lines, and it is noteworthy that Mincle has been shown to suppress Toll-like receptor 4 activation and TLR4 has been proposed to have a central role in the initiation of damaging inflammatory responses during different acute viral infections. In contracts to BGC IMM-101 does not activate TLR4. In a similar manner Mincle suppresses Th17 immune responses that as well have been suggested in coronavirus immunopathology and vaccine-induced immune enhancement.

In another preferred embodiment of the invention, there is provided a method, adjuvant, pharmaceutical composition or use according to the disclosure herein, wherein said human subject at elevated risk of exposure to and/or severity of said viral infection demonstrates a reduction in immunosenescence following administration of said immunomodulator and one or more biologically-active agents, as exhibited by an upregulation of the cellular immune response such as the adaptive immune response, upregulation of the B cell, CD8+ T cell and CD4+ T cell response, and/or increase in T-cell repertoire and/or newly emerging thymic emigrants, optionally wherein the human subject is at least 50, 55, 60, 65, 70, 75, 80, 85 or 90 or more years old.

In a further embodiment of the invention, there is provided a method, adjuvant, pharmaceutical composition or use according to the disclosure herein, wherein said human subject at elevated risk of exposure to and/or severity of said viral infection demonstrates a reduction in inflammaging following administration of said immunomodulator and one or more biologically-active agents, as exhibited by a reduction in chronic (low grade) systemic inflammation, such as reduced levels of pro-inflammatory cytokines n peripheral blood, including TNFalpha or interleukin-6 (IL-6), or other inflammation markers, such as TRAIL, CRP or serum amyloid alpha, optionally wherein the human subject is at least 50, 55, 60, 65, 70, 75, 80, 85 or 90 or more years old.

In a further embodiment of the invention, there is provided a method, adjuvant, pharmaceutical composition or use according to the disclosure herein, wherein said human subject at elevated risk of exposure to and/or severity of said viral infection is immunocompromised, for example, they are suffering from a congenital or acquired immune deficiency, selected from, (i) concurrent disease such as, AIDS, leukemia, lymphoma, (ii) cancer therapy such as exposure to cytotoxic drugs, radiation, or (iii) immunosuppressive therapy such as corticosteroids. It is noted that BCG is contraindicated for such immunocompromised subjects.

In a further embodiment of the invention, there is provided a method, adjuvant, pharmaceutical composition or use according to the disclosure herein, wherein said human subject at elevated risk of exposure to and/or severity of said viral infection, has one or more comorbidity selected from: metabolic disorder, obesity, diabetes, asthma, COPD, hypertension, cardiac disease, arrhythmia, renal disease, liver disease, hematolgic disease, dementia and other neurological disorder, rheumatolgical disease, malnutrition, thrombosis or cancer, optionally wherein said comorbidity is chronic.

In a further preferred embodiment of the invention, there is provided a method, adjuvant, pharmaceutical composition or use according to the disclosure herein, wherein said human subject at elevated risk of exposure to and/or severity of said infection, has one or more tumours or cancer, for example, prostate cancer, liver cancer, renal cancer, lung cancer, breast cancer, colorectal cancer, pancreatic cancer, brain cancer such as glioblastoma, hepatocellular cancer, lymphoma, leukaemia, gastric cancer, cervical cancer, ovarian cancer, thyroid cancer, melanoma, head and neck cancer, skin cancer and soft tissue sarcoma and/or other forms of carcinoma. The tumour may be metastatic or a malignant tumour.

In a further embodiment of the invention, there is provided a method, adjuvant, pharmaceutical composition or use according to any preceding aspect, wherein said human subject at elevated risk of exposure to and/or severity of said infection demonstrates a reduction in dysregulation of the immune system following administration of said immunomodulator and one or more biologically-active agents, before or after said infection exhibited by preventing immune dysregulation and/or hyperactivation evidenced by reduction of an overabundance or unusually high frequency of cell expressing the surface marker(s) CD38+ or CD38+ and HLA-DR+ or PD-1+ and CD38+ (hi) and CD8+ or CD38+ and CD101+ and PD1+ and CD8+.

In a further preferred embodiment of the invention, administration of the non-pathogenic non-viable Mycobacterium comprises oral inhalation, such as via nebuliser, preferably selected from M. vaccae, including the strain deposited under accession number NCTC 11659 and associated designations such as SRL172, SRP299, IMM-201, DAR-901, and the strain as deposited under ATCC 95051 (Vaccae™); M. obuense, M. paragordonae (strain 49061), M. parafortuitum, M. aurum, M. indicus pranii, M. w, M. kyogaense (as deposited under DSM 107316/CECT 9546), M. manresensis, M. tuberculosis Aoyama B or H37Rv, RUTI, z-100 and combinations thereof, preferably the strain of Mycobacterium obuense deposited under the Budapest Treaty under accession number NCTC 13365. In particular, the selected form of M. vaccae is that produced by ANHUII Zhifei long Biopharmaceutical Co., called Vaccae®.

Chinese patent application number CN111265500A discloses a clinical trial as follows:

### 1. Materials and Methods

1.1 Materials and equipment: Mycobacterium vaccae for injection (trade name: microcard, ingredient is Mycobacterium vaccae mycelial protein, Chinese medicine standard S200110003) purchased from Anhui Zhifeilong koma Biopharmaceutical Co., Ltd.; SW- II Jet nebulizer [Gui Xie Zhun 20182540033, Gui Food and Drug Administration Xun No. 20110001].

### 1.2 Method

1.2.1 Research objects and inclusion criteria: Since Feb. 5, 2020, admitted to Nanning Fourth People's Hospital (designated hospital), according to the National Health Commission “New Coronavirus Pneumonia Diagnosis and Treatment Plan (Trial Version 5)”, the diagnosis is ordinary There are 32 patients with type. 1.2.2 Grouping of research subjects: set by random, blind, and control group. There are 18 cases and 14 cases in the conventional control group and the intervention group. The general data of the two groups are comparable. 1.2.3 Treatment distribution and infusion: 1) Apply a computer-generated random plan to assign patients 2) One person was designated to dispense the medicine (the control group was nebulized to inhale 10 ml of normal saline, and the intervention group used 2 microcards dissolved in 10 ml of normal saline and then nebulized inhaled.) 3) The clinical team, researchers, and patients are blinded to the ingredients of the therapeutic drugs (blind method). 4) The conventional treatment control group was treated strictly in accordance with the guidelines+nebulized normal saline inhalation, and the intervention group was based on the conventional treatment control group+nebulized inhalation of microcalorie. Once a day, each time until the liquid was inhaled (approximately 20 minutes), both groups were inhaled 3 sprays of Ventolin before nebulization treatment. 1.2.4 Observation indicators: 1) The primary result of the test was viral nucleic acid in respiratory specimens and blood specimens. 2) The secondary results are chest CT, blood routine, CRP, liver and kidney function, myocardial enzymes, arterial blood gas analysis. Finger pulse oxygen saturation was recorded every hour before nebulization treatment and after nebulization. 3) Severity rate, mortality, length of hospital stay and complications during treatment. 1.2.5 Statistical analysis Kaplan-Meier method was used to analyze the time to negative conversion between the groups, and SPSS 22.0 software was used for statistical analysis. The two-sided test was performed. The test level was α=0.05, and P was considered as statistically significant

1.3 Partial result 1.3.1 Comparison of the time of nucleic acid conversion of throat swabs between the two groups. The intervention group compared with the conventional control group, P<0.05. Compared with the conventional control group (9.143±1.678), the time to negative in the intervention group (5.333±0.800) was P<0.05, suggesting that the time to negative in the intervention group was significantly shorter than that in the conventional treatment group. 1.3.2 Comparison of negative conversion rate, severe disease rate, and death rate between the two groups. The intervention group all turned negative, and 1 case in the conventional treatment group did not turn negative (it has not turned negative for up to 1 month); the intervention group did not turn into a severe case, while 1 case in the conventional treatment group turned into a severe case. There were no deaths.

Preliminary results showed that all patients in the intervention group turned negative within a few days (there is still 1 case in the conventional treatment group that has not turned negative for 1 month), and the time of turning negative was significantly shorter than that in the conventional treatment group. No case became severe or Death, the hospital's recent discharge cases increased significantly, and the rate of cure and discharge quickly exceeded 60% (see the Guangxi dynamic epidemic report on the 25th), and no drug related adverse reactions were found; and, because of the equipment used in the program, the materials and technology are very simple and can be carried out in any medical institution. It embodies the characteristics of simplicity, low price, easy promotion, and suitable for grassroots epidemic prevention and control. As mentioned earlier, inhalation of microcards is mainly used for prevention and treatment by quickly (3-5 days) increasing the level of endogenous interferon in the respiratory tract-lung tissue to inhibit viral RNA replication, reduce cell damage or promote damaged cell repair

Conclusion: This clinical trial initially verified our scientific hypothesis that Mycobacterium vaccae (microcalorie) for nebulized inhalation injection has a good clinical effect in the treatment of COVID-19, and the treatment effect combined with salbutamol sulfate is better, which is beneficial.

In another embodiment of the invention, administration of the non-pathogenic non-viable Mycobacterium comprises intradermal injection of M. indicus pranii, or M. w, marketed by Cadila Healthcare (India), under the brand names Mycidac-C or preferably, Sepsivac. A clinical trial assessing the efficacy of this particular species of mycobacteria, is described in the publication by Jaiswal et al (“Innate Immune Response Modulation and Resistance to SARS-CoV-2 infection: A Prospective Comparative Cohort Study in High Risk Healthcare Workers”; Santa Rani Jaiswal, Anupama Mehta, Gitali Bhagwati, Rohit Lakhchaura, Hemamalini Aiyer, Bakulesh Khamar, Suparno Chakrabarti: medRxiv 2020.10.20.20214965; doi: https://doi.org/10.1101/2020.10.20.20214965). The trial is described as follows:

Methods Thirty-two HCWs from the Department of Blood and Marrow Transplantation and Hematology were administered 0.1 ml Mw (Sepsivac, Cadila Pharmaceuticals, India) intradermally in each arm on day 1 of the study (Mw group) and followed up for 100 days. 64 age matched HCWs from the rest of the hospital were enrolled in a control group. All HCW included in the study had a nasopharyngeal swab evaluated for SARS-CoV-2 by reverse transcriptase-polymerase chain reaction (RT-PCR), on development of symptoms suggestive of COVID-19 or following exposure to an infected person. Body temperature, pulse rate, oxygen saturation and self-reporting of symptoms was evaluated before and after each working day in the Mw group. ‘Exposure’ was defined as close contact with SARS-CoV-2 infected individuals without full protective gear. COVID-19 was graded as mild, moderate or severe as per WHO criteria. Subjects in the Mw group underwent two additional random SARS-CoV-2 specific RT-PCR evaluation 4 weeks apart. The study was approved by the institutional ethics committee.

Results—Overall, 31 out of 96 HCW enrolled had RT-PCR-confirmed COVID-19 infection of which 30 (96.77%) were in control group. Of the 31, who developed COVID-19 infection, four required hospitalization. All belonged to the control group. Despite a greater number of exposures in the Mw group, only one out of 32 (3.13%) subjects had an RT-PCR confirmed mild COVID-19 infection. HR for developing COVID-19 in the control group compared to the Mw group was 19.025 (p=0.0038). Based on this study, the resistance to infection (protective efficacy) provided by Mw was 93.33% (p=0.0001; 95% CI 53.3-99.1). The only side effect noted with Mw was injection site reactions (moderate to severe—4; mild—10), which were self-limiting and did not require any specific management

Discussion—The study period coincided with the peak of the pandemic in New Delhi and the high rate of COVID-19 seen in control group is in line with that reported in similar HCW. The resistance to COVID-19 seen in the Mw group suggests that a TLR2 agonist Mw might be useful in providing protection to subjects at a high risk of exposure. It will be interesting to study its long-term protective efficacy. BCG, another approved immunomodulator is also being evaluated for the prevention of COVID-19, and it will be interesting to study its outcome as there are differences in the innate immune response generated by the two in terms of being a TLR agonist and in ligand presentation. Specific immune changes like upregulation of adaptive natural killer cells are being investigated by our group to understand the immune mechanism responsible for resistance to COVID-19. This study provides an initial proof to the concept of modulating the innate immune response for providing resistance to novel pathogens like SARS-COV2 until the availability of vaccines.

### EXAMPLE 1

An Investigation Into the Influence of Specific Mycobacteria on Responses in Human γδ T-Cell-Cells. γδ T-cell responses were characterised by measuring cytokine production, expression of granzyme B and cytotoxicity against tumour target cells. Results show that T-cells are activated by the mycobacterial preparations heat-killed M. vaccae NCTC 11659, heat-killed M. obuense NCTC 11365 and heat-killed BCG (Danish Strain 1331), as indicated by upregulation of activation marker expression and proliferation. Activated T-cells display enhanced effector responses, as shown by upregulated granzyme B expression, production of the Th1 cytokines IFN-gamma and TNF-alpha, and enhanced degranulation in response to susceptible and zoledronic acid-treated resistant tumour cells. Moreover, T-cell activation is induced by IL-12, IL-1 and TNF-alpha from circulating type 1 myeloid dendritic cells (DCs), but not from type 2 myeloid DCs or plasmacytoid DCs. Taken together, we show that BCG, M. vaccae and M. obuense induce T-cell anti-tumour effector responses indirectly via a specific subset of circulating DCs (Fowler et al., Cancer Immunol Immunother (2012) 61:535-547).

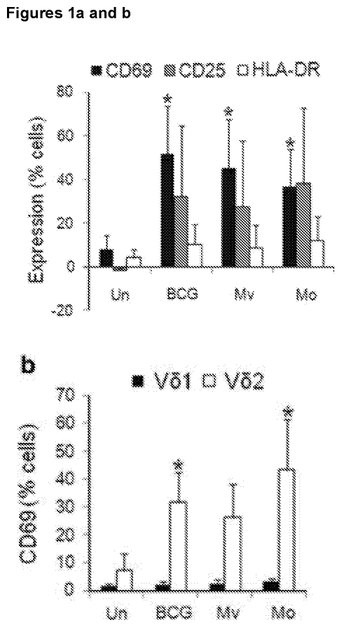

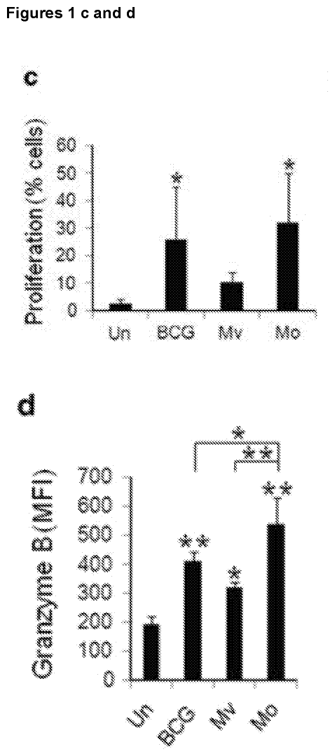

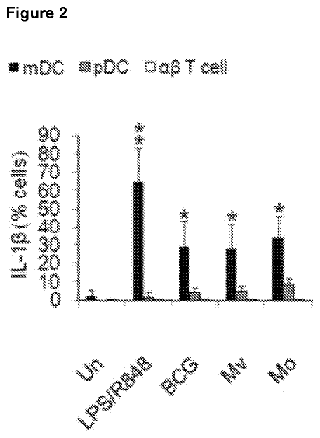

The data also demonstrated the superiority of IMM-101 (M. obuense) over HK BCG in the context of anti-COVID-19 activity and immunosenescence, as seen in FIG. 1. As can be seen, IMM-101-induced proliferation and upregulation of CD69, CD25 and HLA-DR on γδ T-cells, was greater than that observed for BCG, particularly with respect to the V52 subset purported to be involved in anti-SARS-CoV immune surveillance in vivo. The study also showed that IMM-101 activated γδ T-cells enhanced effector responses, upregulated granzyme B expression and enhanced production of TNF-α, to a greater extent compared to BCG.

CD4+ cells were cultured overnight with LPS/R848, heat-killed BCG, M. vaccae (Mv) and M. obuense (Mo). Untreated (un) cells were used as a negative control. IL-12, IL-1 beta and TNF-alpha expression was measured on gated T-cells (CD3+), mDCs (CD3- CD11c+) and pDCs (CD3- CD11c-CD123^{high}). Mean values for n=3 are shown (except TNF-alpha, where n=2). Error bars represent SD. * and ** indicate P values of <0.05 and <0.001, respectively, for statistical comparisons between treated and untreated conditions (see FIG. 2).

Accordingly, the heat-killed mycobacteria demonstrate upregulated IL-1 beta on mDCs, indicative of ‘trained immunity’, and may exhibit a superior immunostimulatory activity in the elderly compared to that for the live attenuated marketed BCG vaccine. Furthermore, it is anticipated there may be reductions in relative risks between administrations of heat-killed vs. attenuated mycobacteria, due to the reduced potential for proliferation/infection.

FIG. 1 description—γδ T-cells within BCG-, M. vaccae- and M. obuense-treated PBMCs produce granzyme B and Th1 cytokines. PBMCs were cultured with heat-killed BCG, M. vaccae (Mv) and M. obuense (Mo) and responses measured within gated T-cells. Untreated (un) cells were used as a negative control. (a) Mean percentages of Vδ1+ and Vδ2+ cells expressing CD69 (at 24 h; n=5), CD25 and HLA-DR (both at 48 h; n=3) are shown. (b) Mean percentages of Vδ1+ and Vδ2+ cells expressing CD69 CD69 (at 24 h; n=3). (c) CFSE+ PBMCs were cultured for 6 days with mycobacteria and proliferation measured within the γδ T-cell compartment. Mean percentages of γδ T-cells proliferating are shown (n=6). (d) Mean fluorescent intensities (MFI) of granzyme B expression within γδ T-cells (at 24 h; n =3).

### EXAMPLE 2

IMM-101 VACCINATION TO REDUCE THE IMPACT OF SARS-COV-2 (COVID-19) INFECTION IN HEALTH CARE WORKERS AFTER HIGH-RISK EXPOSURES

Dosage: A single 0.1 mL intradermal injection of IMM-101 (10 mg/mL)

Administration: IMM-101 Dosing Regimen: The treatment regimen with IMM-101 will be one dose given on day 1 with a follow up booster dose given on day 14.

Note: IMM-101 is given via intradermal injection into the skin overlying the deltoid muscle, with the arm being alternated between each dose. Local skin reactions are expected but, in the event of an injection site reaction of Grade 3 (severe) as measured by the NCI CTCAE v5.0, at the discretion of the Investigator, participants may be administered a half dose of IMM-101 (i.e., a single 0.05 mL intradermal injection of IMM-101) or the dosing interval may be increased or both.

Study period: 12 months

### Objectives:

The assumption made is that IMM-101 compared to the matching placebo (normal saline), will reduce both the number of cases of COVID-19 (and therefore possibly increase the number of asymptomatic SAR-CoV-2 infections) and the number of severe cases of COVID-19. Thus, IMM-101 would be able to shift the “severity of COVID-19” curve down (generally reduce the severity of the symptoms in healthcare workers).

### Primary Objectives:

1. To determine if IMM-101 administration compared with matching placebo (normal saline), reduces the incidence of symptomatic and asymptomatic COVID-19 disease (Outcome) determined by RT-PCR and serology testing measured over the 6 months following randomization in healthcare workers exposed to SAR-CoV-2 (participants).
2. To determine if IMM-101 administration compared to matching placebo (normal saline) reduces the incidence of severe COVID-19 infection resulting in hospitalization and/or admission to intensive care units or death measured over the 6 months following randomization (Time) in healthcare workers exposed to SARS-CoV02.

### Secondary Objectives:

To evaluate the safety of IMM-101 vaccination in adult health care workers. Participants will be closely monitored for any occurrence of adverse events or serious adverse events during the study. Regular in-person visits will be scheduled to capture the frequency, intensity, and relationship to study drug of (serious) adverse events, any abnormal clinical and/or biological signs. Body temperature will also be collected in all follow-up visits.
To determine if IMM-101 compared to placebo prolongs the time to first SAR-CoV-2 proven respiratory illness (Outcome) measured over 6 months following randomization (Time) in healthcare workers exposed to SARS-CoV-2 (participants).
To determine if IMM-101 compared to placebo prolongs the time to first SAR-CoV-2 proven clinical features of COVID-19 (Outcome) measured over 6 months following randomization (Time) in healthcare workers exposed to SARS-CoV-2 (participants).
To determine if IMM-101 compared to placebo reduces severity of COVID-19 disease (Outcome) measured over 6 months following randomization (Time) in healthcare workers exposed to SARS-CoV-2 (participants)
To determine if IMM-101 compared to placebo reduces the incidence and severity of febrile respiratory disease measured over 6 months following randomization (Time) in healthcare workers exposed to SARS-CoV-2 (participants).
To determine if IMM-101 compared to placebo reduces unplanned absenteeism (days off work) measured over 6 months following randomization (Time) in healthcare workers exposed to SARS-CoV-2 (participants).
To determine if IMM-101 compared to placebo reduces unplanned absenteeism (days off work) due to disease measured over 6 months following randomization (Time) in healthcare workers exposed to SARS-CoV-2 (participants).

### Exploratory Objectives

To determine in a subgroup of adults with recurrent cold sores whether IMM-101 administration compared to placebo reduces herpes simplex recurrences (such as cold sores).
To determine in a subgroup of adults with recurrent urinary tract infections (UTIs) whether IMM-101 administration compared to placebo reduces UTIs recurrences.

### Endpoints:

### Primary Endpoint:

Number of participants with COVID-19 disease defined as fever plus at least one sign or symptom of respiratory disease including cough, shortness of breath, respiratory distress/failure, runny/blocked nose (using self-reported questionnaire) or less common signs and symptoms of COVID-19 infection including headache, muscle pain, abdominal pain, diarrhea, sputum production, anosmia, stroke, and sore throat, plus a positive SARS-Cov-2 test (PCR or serology) over the 6 months following randomization.
Number of participants who were admitted to hospital, ICU and/or died (using self-reported questionnaire and/or medical/hospital records) in the context of a positive SARS-CoV-2 test, over the 6 months following randomization.

### Secondary Endpoint:

These will be assessed over the 6 months following randomization unless otherwise indicated.
Participants will be closely monitored for any occurrence of adverse events or serious adverse events during the trial. Regular in-person visits will be scheduled to capture the frequency, intensity, and relationship to study drug of (serious) adverse events. Body temperature will also be collected in all follow-up visits.
Number of participants with COVID-19 disease, days unable to work, days confined to bed, days with symptoms in any episode of illness that meets the definition of COVID-19 disease, pneumonia, need for oxygen therapy, admission to critical care, need to mechanical ventilation, Bilevel Positive airway pressure (BiPAP), Extra Corporal Membrane Oxygenation (ECMO).
Number of episodes of COVID-19/febrile respiratory illness
Time to first symptom of COVID-19/febrile respiratory illness
Number of deaths
Number of days of unplanned absenteeism
Number of days of unplanned absenteeism due to disease
Type and severity of local and systemic adverse event over the 6 months following randomization

### Exploratory Endpoint:

Number of participants with, number of episodes, and time to first recurrence of herpes simplex (cold sores and genital), and/or UTIs.

### Methodology:

This prospective double-blind randomized trial will study the safety, tolerability, and efficacy of IMM-101 in qualified health care workers.

Individuals who have consented to participate and met the study criteria, will be randomized to either IMM-101 or placebo (normal saline) to be administered as an intradermal injection on day 1 and a booster dose on day 14. All study individuals will follow the same visit and assessment schedule. Following study recruitment, participants will be scheduled to be assessed in person during treatment at days 1, 7, 14, 21, 35, 60, 90, 120, 150 and 180. An independent Study Monitoring Committee (SMC) will review study conduct and unblinded safety data throughout the study and make recommendations as appropriate.

Number of Subjects (planned): 1200 individuals will be enrolled into the study with half enrolling in the active treatment study and the other half, will be entering the matching placebo group.

### Diagnosis and Main Criteria for Inclusion:

1. Adult male and female healthcare workers 18 to 65 years of age upon study consent
2. Hospitalists or health care workers from Emergency Departments, on COVID19 wards or Intensive Care Units/hospitalists (this study is not restricted to clinicians or nurses and includes all individuals who work within the specified areas of the participating hospitals during the COVID-19 outbreak)
3. Participant in direct patient care in the ER or of COVID-19 positive patients on the medical floor or ICU; or significant unprotected high-risk exposures to patients who have been confirmed to have symptomatic COVID-19 infection
4. Afebrile with no constitutional symptoms
5. Negative RT-PCR prior to initial study dosing
6. Willing and able to comply with scheduled visits, treatment plan, and other study procedures
7. Willing to provide blood for future serology and research
8. Evidence that participant has signed and dated informed consent document indicating that the subject has been informed of all pertinent aspects of the study and agreed prior to initiation of any subject-mandated procedures

### Major Exclusion Criteria:

Has a vaccine contraindication
Fever or generalized skin infection (can be enrolled when cleared);
Known condition that leads to suppressed immune system
Receiving medical treatment that is immunosuppressive in the last 6 months e.g. Systemic corticosteroids (≥20 mg for ≥2 weeks), non-biological immunosuppressant (also known as DMARDS'), biological agents (such as monoclonal antibodies against tumor necrosis factor (TNF)-alpha).
People with congenital cellular immunodeficiency's including specific deficiencies of the interferon-gamma pathway
People with known malignancies involving bone marrow or lymphoid system
People with any serious underlying illness (e.g. Malignancy)
People with uncontrolled cardiovascular disease, hypertension, diabetes, and/or chronic respiratory disease and/or people with immune-compromised conditions
Known or suspected HIV infection even if they are asymptomatic or have normal immune function (due to the risk of disseminated mycobacterial infection)
People with active skin disease such as eczema, dermatitis or psoriasis at or near the site of vaccination (a different site other than the left arm can be chosen if necessary)
Women who are pregnant or breast feeding
A live vaccine administered two months prior to treatment or require a live vaccine to be administered within the month following randomization
Previous active TB disease
BCG vaccine given within the last 5 years
1. Previously had SARS-CoV-2 positive test result
2. Positive RT-PCR or serology at the time of screening
3. Participation in other investigational clinical studies involving any investigational product within 30 days prior to Day 1 including other investigational clinical studies involving interventional products being used as prophylaxis for SARS-CoV-2 within 30 days prior to Day 1.
4. Unwilling to practice acceptable methods of birth control (both males who have partners of childbearing potential and females of childbearing potential) during Screening, while being administered the study drug, and for at least 30 days after the last dose of study drug.
5. Has a chronic liver disease or cirrhosis, including hepatitis B and/or untreated hepatitis
6. Untreated or uncontrolled active bacterial, viral or fungal infection
7. Known or suspected active drug or alcohol abuse, per investigator judgment
8. Known hypersensitivity to any component of the study drug

### Duration of Treatment:

Study drug will be administered on day 1 and on day 14

### Criteria for Evaluation:

Efficacy: Incidence rate of symptomatic and asymptomatic COVID-19 infections determined by RT- PCR and serology testing

Safety: Clinical and biological measurements, adverse events, and serious adverse events.

### Statistical Methods:

### Sample Size:

A total sample of 1200 individuals (600 in each arm) would provide at least 80% power to show that IMM-101 vaccination is non-inferior to placebo and could potentially reduce the incidence rate of COVID-19 infection by 50%.

The power calculation, which was based on the Farrington-Manning test for non-inferiority assumes the following:
One-sided Type I error rate of 0.05
Non-inferiority margin of 0.02
Anticipated COVID-19 incidence rate of 4% in the placebo group
Anticipated COVID-19 incidence rate of 2% in the clinical arm
Loss to follow-up rate of 20%

### Primary Analysis of Efficacy:

The proportion of people who become infected with SARS-CoV-2 during the study period will be compared between the treatment (IMM-101) and the placebo (normal saline) groups. In addition, the potential effect of IMM-101 on COVID-19 free survival time will also be explored by utilizing appropriate statistical methods. An intent-to-treat approach will be used throughout the analysis with the placebo group as the referent and a p-value <0.05 will be considered statistically significant.

### Example 3

IMMUNIZATION WITH IMM-101 FOR THE PREVENTION OF SEVERE RESPIRATORY AND COVID-19 RELATED INFECTIONS IN CANCER PATIENTS AT INCREASED RISK OF EXPOSURE

Dosage: Three doses of IMM-101 through intradermal injection consisting of one 0.1 mL does and two 0.05 mL doses.

### Administration:

IMM-101 Dosing Regimen: The treatment regimen with IMM-101 will be one 1.0 mg (=0.1 mL) dose given on Day 0, followed by a second dose of 0.5 mg (=0.05 mL) on Day 14 (−3/+5 days), and a third dose of 0.5 mg (=0.05 mL) on Day 45 (+1-14 days).

Note: IMM-101 is given via intradermal injection into the skin overlying the deltoid muscle, with the arm being alternated between each dose. Local skin reactions are expected but, in the event of an injection site reaction of Grade 3 (severe) as graded by the NCI CTCAE v 5.0, at the discretion of the Investigator the dosing interval may be increased.

Study Period: The study will continue until all patients have completed a minimum of 12 months of follow-up.

### Objectives:

To investigate the effectiveness of IMM-101 at preventing “influenza-like illnesses” (ILI), as defined by the WHO, OR a confirmed viral bacterial respiratory infection (via microbiology or radiography), AND that results in a change or delay in a priori planned cancer treatment or requirement for an unscheduled medical assessment (i.e. emergency room visit, family physician assessment, etc.) hospitalization, or death compared to control subjects.

### Primary Objectives:

1. To determine if IMM-101 administration compared with control patients (observation), reduces the rate of “flu-like illness” which includes WHO definition of ILI [Fitzner 2018] or confirmed viral/bacterial respiratory infection.
2. To determine if IMM-101 administration compared with control patients (observation) results in a change or delay in cancer treatment or requirement for an unscheduled medical assessment (i.e. emergency room visit, family physician assessment, etc.), hospitalization, or death.

### Secondary Objectives:

To compare between IMM-101 vaccinated patients and control patients the incidence of documented COVID-19 infection (confirmed by any Health Canada approved COVID-19 test). Both symptomatic and asymptomatic infections will be documented.
To compare between IMM-101 vaccinated patients and control patients the rate of severe COVID-19 infection defined as a confirmed COVID-19 infection leading to hospitalization, ICU admission or death.
To compare between IMM-101 vaccinated patients and control patient the number of events that meet the definition of the primary endpoint, as measured within the one-year follow-up (patients may meet the primary endpoint more than once and be counted multiple times).
To compare between IMM-101 vaccinated patients and control patients the incidence of COVID-19 seroconverted patients between baseline, 3 months, 6 months and 12 months.
To compare between IMM-101 vaccinated patients and control patients the incremental cost-effectiveness ratio (ICER) (in the unit of CAD$ per life-years gained).
To compare between IMM-101 vaccinated patients and control patients if failure-free survival (FFS), as time from enrolment to recurrence or progression declared by the investigator on the basis of objective standard evaluation consistent with the disease site (e.g. cross sectional imaging, serum tumour markers, etc.) or death.
To compare between IMM-101 vaccinated patients and control patients the overall survival (OS), as time from enrolment to death from any cause.
To compare between IMM-101 vaccinated patients and control patients, incidence, frequency, and severity of AEs considered possibly, probably or definitely related to receipt of IMM-101.
To compare between IMM-101 vaccinated patients and control patients the incidence and frequency of local injection site reactions subsequent to IMM-101 administration.
To compare between IMM-101 vaccinated patients and control patients the incidence and duration of ICU admission related to documented COVID-19 infection.

### Exploratory Objectives

To conduct correlative studies assessing the activation of an innate and subsequent adaptive immune response to IMM-101 in the treatment arm of the study, including changes in PBMC's and plasma cytokines in IMM-101 immunized and in some cases, non-immunized patients over time. Including (i) baseline immune status of each patient and how this changes over time; (ii) immunological markers associated with IMM-101 immunization, changes in innate and adaptive immunity markets and host genomic factors; (iii) analysis of innate and adapted (cellular and humoral) immune response to SARS-CoV-2 or any other infectious agent, should a patient be diagnosed as infected on study follow-up; and (iv) number of outbreaks of herpes simplex cold sores, genital herpes and herpes zoster.

### Endpoint:

### Primary Endpoint:

The primary endpoint is a composite endpoint and includes: WHO definition of ILI [Fitzner 2018] OR confirmed viral/bacterial respiratory infection AND results in a change or delay in cancer treatment or requirements for an unscheduled medical assessment (i.e. emergency room visit, family physician assessment, etc.), hospitalization, or death.
The rate of “flu-like illness” will be calculated for each arm of the study as the proportion of randomized patients who experience this outcome over the duration of the study.
The incidence of documented COVID-19 infection, the rate of severe respiratory and COVID-19 infections, and the incidence of seroconverted patients between baseline, 6 months and 12 months will be analysed similarly as the primary endpoint.

### Secondary Endpoint:

Linkage of clinical trial population to provincial administrative data will allow for the ascertainment of other clinically relevant secondary and exploratory endpoints in the context of the COVID-19 pandemic. Such linkage will allow for ascertainment of resource utilization and costs from the public health care perspective to enable the assessment of the cost-effectiveness of IMM-101.
If a patient meets the criteria for the primary endpoint more than once, each event will be captured for a secondary endpoint but not for the purposes of evaluating the primary outcome.

### Methodology:

This multi-centre, open-label, randomized phase III trial will compare immunization with IMM-101 versus observation for the prevention of severe respiratory and COVID-19 related infections in cancer patients at increased risk of exposure.

Individuals who have consented to participate and met the study criteria, will be randomized to either IMM-101 (ARM 1) or observation (ARM 2). Patients enrolled on ARM 1 will receive three doses of IMM-101 on days 0, 14, and 45. Please refer to Section 7.1.1 for dosing details. The patients randomized to the IMM-101 arm will be required to attend the immunization clinic in person to receive the dose of IMM-101. The patients enrolled to the observation arm will have a virtual visit on the same day, where in the same information and questions will be collected.

Number of Subjects (planned): 1500 patients will be enrolled into the study with half enrolling in the active treatment study and the other half, will be entering the observation group.

### Diagnoses and Main Criteria for Inclusion:

1. Patients must be undergoing (or be planned to undergo) active treatment for one or more solid malignancy, lymphoma or myeloma, requiring them to present to the hospital or cancer clinic at least twice/month for assessments and/or treatments, anticipated for at least 3 months
2. Patients must have one or more of the following risk factors [CDC 2019] for severe COVID-19 infection:
3. Age >65 years old;
4. Hypertension (on medication);
5. Type 1 or 2 Diabetes (on medication);
6. A relevant chronic condition as per the investigator based on the medical record, including: heart (e.g. heart failure, coronary artery disease, congenital heart disease, cardiomyopathies, and pulmonary hypertension); lung (e.g. chronic obstructive pulmonary disease (COPD including emphysema and chronic bronchitis), moderate to severe asthma, idiopathic pulmonary fibrosis and cystic fibrosis); liver cirrhosis; serious kidney disease requiring dialysis;
7. Receiving systemic therapy (such as cytotoxic chemotherapy, immunotherapy or targeted agents excluding single agent hormonal therapy);
8. Body Mass Index >40; and
9. Living in a nursing home or long-term care facility.
10. Patient must have a life expectancy of >6 months as assessed by the investigator
11. Patient must have an ECOG Performance Status 2
12. Patient has adequate organ function appropriate for the therapy the patient is planned to receive in the opinion of the investigator and based on local assessment and practices
13. Patient is aged 18 years
14. Patient has agreed to receive pneumococcal vaccination and a seasonal influenza vaccination in accordance with Canadian Guidelines [Government of Canada, 2019].
15. Patient is able (i.e. sufficiently fluent) and willing to complete the health utility questionnaires in either English or French. The baseline assessment must be completed within required timelines, prior to enrolment. Inability (lack of comprehension in English or French, or other equivalent reason such as cognitive issues or lack of competency) to complete the questionnaires will not make the patient ineligible for the study. However, ability but unwillingness to complete the questionnaires will make the patient ineligible.
16. Patient consent must be appropriately obtained in accordance with applicable local and regulatory requirements. Each patient must sign a consent form prior to enrolment in the trial to document their willingness to participate.
17. Patient must be willing to provide identifying information including provincial health insurance number to facilitate data linkage and follow up.
18. Patients must be accessible for treatment and follow-up. Investigators must assure themselves the patients enrolled on this trial will be available for complete documentation of the treatment, adverse events, and follow-up.
19. Women/men of childbearing potential must have agreed to use a highly effective contraceptive method throughout the treatment period and for at least 3 months after discontinuation of treatment. A woman is considered to be of “childbearing potential” if she has had menses at any time in the preceding 12 consecutive months. In addition to routine contraceptive methods, “effective contraception” also includes heterosexual celibacy and surgery intended to prevent pregnancy (or with a side-effect of pregnancy prevention) defined as a hysterectomy, bilateral oophorectomy or bilateral tubal ligation, or vasectomy/vasectomized partner. However, if at any point a previously celibate patient chooses to become heterosexually active during the time period for us of contraceptive measures outlined in the protocol, he/she is responsible for beginning contraceptive measures.

### Major Exclusion Criteria:

Patient previously received treatment with IMM-101.
Patient cannot have either at present or in the past, a positive test for COVID-19 infection. If a patient has been tested for COVID-19, result must be confirmed as negative prior to enrolment.
Patient cannot have experienced “flu-like symptoms” within 14 days prior to enrolment, including fever, extreme fatigue, new or worsening cough, myalgias, new or worsening dyspnea, and/or sputum production.
Patient is receiving concomitant treatment with another investigational product with another investigational product or has received such treatment within the 3 weeks prior to enrolment.
Patient has any co-existing active infection that, in the opinion of the Investigator, may increase the risk associated with study participation or study drug administration, impair the ability of the subject to receive protocol therapy, interfere with the patient's participation for the full duration of the study, or is not in the best interest of the patient to participate.
Patient has previously experienced an allergic reaction to any mycobacterial product, including the BCG vaccine.
Patients with superficial bladder cancer or any other condition currently receiving or planned to be treated with BCG.
Patient has a known history of Human Immunodeficiency Virus (HIV) (HIV ½ antibodies) or a known history of or is known to have a positive test for Hepatitis B (HBsAg reactive) or Hepatitis C (HCV RNA [qualitative]).
Patients with prior or concurrent leukemia.
Patient has had a prior bone marrow transplant.
Patient is pregnant or breast-feeding.
Patient has documented history of clinically severe autoimmune disease or a syndrome that requires systemic steroids or immunosuppressive agents. This includes patient requiring systemic treatment with either corticosteroids (>10 mg daily prednisone equivalent, or depot corticosteroids in the 6 weeks before enrolment) or immunosuppressant drugs (such as azathioprine, tacrolimus, cyclosporine, etc.) within the 14 days prior to enrolment or a reasonable expectation that the patient may require such treatment during the course of the study. Inhaled or topical or inter-articular steroids, and adrenal replacement steroid doses 10 mg daily prednisolone equivalent, are permitted in the absence of active autoimmune disease. Steroids used for premedication prior to chemotherapy or as part of a chemotherapy regimen are allowed.

### Duration of Treatment:

Study drug will be administered on Day 0, Day 14 and Day 45.

### Criteria for Evaluation:

Efficacy: The incidence of documented COVID-19 infection, the rate of severe respiratory and COVID-19 infections, and the incidence of seroconverted patients between baseline, 6 months and 12 months will be analysed similarly as the primary endpoint. The number of events that meet the definition of the primary endpoint between two treatment arms will be compared by a Wilcoxon test.

A Cochran-Mantel-Haenszel test adjusting for the stratification factors at the time of enrolment will be used as the primary method to compare the “flu-like illness” rates between two arms at all analysis time points, with appropriate alpha-spending adjustment for interim analyses as performed.

Safety: Only adverse events on study considered to be possibly, probably or definitely related to receipt of IMM-101 will be collected. Patients on Arm 1 will be evaluable for adverse event evaluation from receipt of IMM-101 (Day 0).

### Statistical Methods:

### Sample Size:

A total sample size of 1500 patients (750 in each group will enable detection of a 50% reduction in the incidence of COVID-19 related infection by the treatment of IMM-101 (i.e. from 6% to 3%) with 80% power and at a two-sided 0.05 level.

### Primary Analysis of Efficacy:

The patients in two treatment groups will be compared in the incidence of documented COVID-19 infection, the rate of severe respiratory and COVID-19 infections, the incidence of seroconverted patients between baseline, 6 months and 12 months, the incremental cost-effectiveness ratio, investigator reported objective cancer progression and overall survival.

The incidence, frequency, and severity of adverse events graded using the NCI Common Toxicity Criteria Version 5.0 and considered possibly, probably or definitely related to receipt of IMM-101 will be documented.

1-75. (canceled)

76. A method of treatment or prevention of a viral infection and/or the symptoms thereof in a subject, comprising administering to the subject an immunomodulator, wherein the immunomodulator comprises non-pathogenic non-viable Mycobacterium obuense, and wherein the viral infection is an infection caused by a coronavirus.

77. The method according to claim 76, wherein the coronavirus is SARS-CoV-2.

78. The method according to claim 76, wherein the coronavirus is SARS-CoV.

79. The method according to claim 76, wherein the coronavirus is MERS-CoV.

80. The method according to claim 76, wherein the non-pathogenic non-viable Mycobacterium obuense is the rough variant.

81. The method according to claim 76, wherein the non-pathogenic non-viable Mycobacterium obuense is the strain NCTC 13365.

82. The method according to claim 76, wherein the non-pathogenic non-viable Mycobacterium obuense is administered via a parenteral, oral, sublingual, nasal or pulmonary route.

83. The method according to claim 82, wherein the parenteral route is selected from subcutaneous, intradermal, subdermal, intraperitoneal or intravenous injection, optionally peritumoral, perilesional, intralesional or intratumoral injection, wherein said human subject has one or more tumours.

84. The method according to claim 83, wherein the parenteral route is intradermal injection.

85. The method according to claim 84, wherein the immunomodulator is administered in an intradermal injection via a microneedle device comprising a plurality of needles, optionally wherein said microneedles are hollow.

86. The method according to claim 76, wherein the immunomodulator is prepared for administration within a single-use pre-filled syringe or multi-dose applicator or jet injector.

87. The method according to claim 76, wherein the amount of non-pathogenic non-viable Mycobacterium obuense administered is from 10⁷ to 10⁹ cells per unit dose.

88. The method according to claim 76, wherein the non-pathogenic non-viable Mycobacterium obuense modifies a cellular immune response.

89. A method of treatment or prevention of an infection and/or the symptoms thereof in a human subject at elevated risk of exposure to and/or severity of said infection, comprising administering to the subject an adjuvant in conjunction with one or more biologically-active agents, wherein the adjuvant comprises a non-pathogenic non-viable Mycobacterium, wherein the infection is an infection caused by a virus, bacteria, protozoa or fungus, preferably wherein the virus is a coronavirus.

90. The method according to claim 89, wherein said one or more biologically-active agent is a therapeutic drug, nutraceutical, cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof.

91. The method according to claim 90, wherein said biologically-active agent is an antigen or antigenic determinant.

92. The method according to claim 91, wherein said antigen or antigenic determinant is specific for, targeted to and/or derived from a coronavirus, or is a vehicle used for delivery thereof, such as an adenoviral vector, vaccinia vector, plasmid vector, optionally live attenuated; mRNA, a modified mRNA, or a stabilized mRNA; viral replicase, spike protein, spike fragment, envelope protein, membrane protein, nucleocapsid protein, subunit of a spike protein, receptor binding domain (RBD) of the subunit of a spike protein, or a functional fragment or variant thereof.

93. The method according to claim 89, wherein the coronavirus is SARS-CoV-2.

94. The method according to claim 89, wherein the coronavirus is SARS-CoV.

95. The method according to claim 89, wherein the coronavirus is MERS-CoV.

96. The method according to claim 89, wherein the non-pathogenic non-viable Mycobacterium is selected from M vaccae, including the strain deposited under accession number NCTC 11659 and associated designations such as SRL172, SRP299, IMM-201, DAR-901, and the strain as deposited under ATCC 95051 (VACCAE™); M. obuense, M. paragordonae (strain 49061), M. parafortuitum, M. aurum, M. indicus pranii, M.w, M. manresensis, M. kyogaense (as deposited under DSM 107316/CECT 9546), M. tuberculosis Aoyama B or H37Rv, RUTI or Z-100 and combinations thereof, preferably the strain of Mycobacterium obuense deposited under the Budapest Treaty under accession number NCTC 13365.

97. The method according to claim 89, wherein the non-pathogenic non-viable Mycobacterium is a rough variant and/or whole cell.

98. The method according to claim 89, wherein the non-pathogenic non-viable Mycobacterium obuense is the strain deposited under the Budapest Treaty under accession number NCTC 13365.

99. The method according to claim 76, wherein the non-pathogenic non-viable Mycobacterium is administered via a parenteral, oral, sublingual, nasal or pulmonary route.

100. The method according to claim 89, wherein the non-pathogenic non-viable Mycobacterium and/or one or more biologically-active agents are administered in the same composition via the same route or in separate compositions each via a different route, optionally at the same time or different times.

101. The method according to claim 99, wherein the parenteral route is selected from subcutaneous, intradermal, subdermal, intraperitoneal or intravenous injection, optionally peritumoral, perilesional, intralesional or intratumoral injection, wherein said human subject has one or more tumours.
